(19) United States
(12) Patent Application Publication
(10) Pub. No.: US 2024/0122878 A1
(43) Pub. Date: Apr. 18, 2024

(54) MODIFIED RELEASE FORMULATIONS OF METHADONE AND ITS ISOMERS, ESMETHADONE AND LEVOMETHADONE AND DERIVATIVES

(71) Applicants: Paolo L. Manfredi, Miami Beach, FL (US); Charles E. Inturrisi, New York, NY (US)

(72) Inventors: Paolo L. Manfredi, Miami Beach, FL (US); Charles E. Inturrisi, New York, NY (US); Sara De Martin, Vigonza (IT); Andrea Mattarei, Perarolo di Vigonza (IT); Gianfranco Pasut, Padova (IT)

(21) Appl. No.: 18/572,542

(22) PCT Filed: Jun. 28, 2022
(86) PCT No.: PCT/US2022/035255

§ 371 Date: Dec. 20, 2023

Related U.S. Application Data
(60) Provisional application No. 63/216,647, filed on Jun. 30, 2021.

(51) Int. Cl. A61K 31/137; A61K 9/00; A61K 9/20; A61K 9/28

(52) U.S. Cl. CPC A61K 31/137; A61K 9/0053; A61K 9/2018; A61K 9/2027; A61K 9/2054; A61K 9/2059; A61K 9/2813; A61K 9/2846; A61K 9/2853

(57) ABSTRACT

A modified release formulation of esmethadone. levomethadone. or racemic methadone, wherein the formulation has a Cₘₐₓ at least 10% lower, and/or a Tₘₐₓ at least 10% longer, and/or a Cₘᵢₙ (C_{trough}) at least 10% higher compared to the Cₘₐₓ, Tₘₐₓ, and Cₘᵢₙ of an immediate release formulation including the same dose of esmethadone, levomethadone, or racemic methadone.

### CROSS-REFERENCE TO RELATED APPLICATION

This application claims priority to, and the benefit of the filing date of, U.S. Patent Application Ser. No. 63/216,647, filed on Jun. 30, 2021, the disclosure of which is incorporated by reference herein in its entirety.

### FIELD OF THE INVENTION

The present invention generally relates to methadone and its isomers and derivatives, and more specifically to modified release formulations including methadone or its isomers or derivatives.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Drug delivery optimization is the process of administering an active pharmaceutical ingredient (API) in the best formulation in order to safely obtain the required therapeutic effect. The oral route is often the preferred administration method and has a high degree of compliance. The ideal oral formulation reaches therapeutic concentration rapidly and maintains the concentration within the therapeutic window for the desired treatment period, without fluctuations that could potentially cause toxic or ineffective concentrations. However, this objective is sometimes difficult to obtain because there are several variables that must be considered. Among these variables are characteristics of drug absorption, metabolism, excretion, half-life, dose and the frequency of dosing and pharmacodynamic characteristics. All of these characteristics should be carefully considered based on pharmacokinetic and pharmacodynamic preclinical and clinical studies, including the stability of the drug. With the oral route of administration, gastro-intestinal (GI) motility is also a factor. Certain drugs, including methadone and its isomers, change GI motility. In vivo elimination half-life of drugs and their therapeutic index are significant parameters in deciding the drug dose and the dosing frequency. Frequency of dosing is inversely correlated with compliance and therefore increased dosing frequency may result in decreased efficacy [Cristina Maderuelo, Aránzazu Zarzuelo, José M Lanao. Critical factors in the release of drugs from sustained release hydrophilic matrices. J. Control. Release, 154 (1) (2011), pp. 2-19]. The common limitations associated with conventional immediate release of oral formulations are frequent administration, fluctuations in plasma concentration (especially for drugs with a dosing interval longer than their half-life), adverse effect in the case of drugs with a narrow therapeutic window. These side effects are usually dose-dependent and may be worse at at Cₘₐₓ, and may be worse with drugs with a short Tₘₐₓ. Additionally, immediate release formulations may show decreased efficacy at Cₘᵢₙ (C_{trough}), or when the frequency of dosing is increased, this potentially causes decreased patient compliance and again decreased efficacy.

The basic rationale (and thus typical procedure) when designing oral controlled-oral dosage forms is to modify the pharmacokinetics and thus the pharmacodynamics of an active moiety by modifying its absorption through the GI tract. Modified-release formulations can be developed to offer an effective means to optimize the bioavailability and plasma concentrations of the drug, thereby improving safety and efficacy. In these formulations, the rate of GI drug release is predicted and controlled. These modifications are attained by a special formulation design and a manufacturing method. One such modified release formulation is the controlled-release system. This system has three main features: (1) it achieves modified release of the drug over time; (2) it facilitates constant levels of drug at the target site necessary for therapeutic effects; and (3) it delivers the drug systemically or to a specific site at a predetermined rate for a specific period of time [Gautam Singhvi, Ravi Ukawala, Harish Dhoot, Suresh Jain. Design and characterization of controlled release tablet of metoprolol. J. Pharm. Bioall. Sci., 4 (5) (2012), p. 90, 10.4103/0975-7406.94152]. By modifying the release rate to decrease the rate of absorption, the dosing interval can be increased, or the drug will have a better PK profile because of lower Cₘₐₓ and higher Cₘᵢₙ (C_{trough}), considering the transit time through the GI tract. The result of this approach is less fluctuation in plasma drug concentration, potentially leading to improvement of the treatment outcome compared to immediate-release formulations. In fact, to maintain effective steady state plasma levels of the drug, a conventional, immediate-release drug oral dosage formi generally requires administration at frequent intervals, with risk of errors and decreased compliance in the drug regime.

Methadone, due to its long half-life, is considered a long-acting agonist of the μ opioid receptor. Methadone is marketed in most of the world as a racemic 50/50 mixture containing two enantiomers: (1) esmethadone, also known as dextromethadone, d-methadone, S-methadone, and (+)-methadone; and (2) levomethadone, also known as R-methadone, 1-methadone, and (−)-methadone. The opioid agonist activity of racemic methadone is due to levomethadone, while esmethadone is the opioid-inactive dextro-isomer and is a low-affinity, low potency blocker of the N-methyl-D-aspartate receptors (NMDARs). NMDAR are glutamate gated ion channels playing a pivotal role in the regulation of synaptic function in the central nervous system (CNS). Esmethadone exerts its blocking activity on NMDAR by binding the MK-801 site with low-micromolar half-maximal inhibitory concentration (IC₅₀) value [Gorman A L, Elliott K J, Inturrisi C E. The d- and l-isomers of methadone bind to the non-competitive site on the N-methyl-D-aspartate (NMDA) receptor in rat forebrain and spinal cord. Neurosci Lett. 1997 Feb 14:223(1):5-8. doi: 10.1016/80304-3940(97)13391-2. PMID: 9058409]. Esmethadone shares the ability of binding to MK-801 site within the channel pore with other known uncompetitive NMDA receptor antagonists, such as phencyclidine, phenylcyclohexylpiperidine (PCP), ketamine and dextromethorphan, among others. As stated, esmethadone is virtually opioid-inactive, having 20-fold lower affinity for μ opioid receptors with respect to levomethadone (Codd. E. E., R. P. Shank, J. J. Schupsky, and R. B. Raffa. 1995. ‘Serotonin and norepinephrine uptake inhibiting activity of centrally acting analgesics: structural determinants and role in antinociception’. J Pharmacol Exp Ther, 274: 1263-70), and does not appear to contribute in a meaningful way to the opioid effects of racemic methadone, which are due to its enantiomer levomethadone (Drug Enforcement Administration (DEA). Diversion Control Division. Drug & Chemical Evaluation Section. Methadone. December 2019).

https://www.deadiversion.usdoj.gov/drug chem info/methadone/methadone.pdf-search=methadone).

The present inventors have been developing racemie methadone and its isomers (esmethadone and levomethadone) and their derivatives for various indications and have performed several novel preclinical studies in vitro and in vivo, as well as clinical investigations in patients to assess activity, toxicity and pharmacological features of racemic methadone and its isomers, leading to an advance of the scientific knowledge for methadone, its isomers, and its derivatives for several decades. As far as its therapeutic use is concerned, racemic methadone is a synthetic opioid with over 70 years of clinical uses, mainly for the treatment of opioid use disorder and for the treatment of pain. Furthermore, tablets and/or syrups containing only levomethadone have been authorized in some countries, for the treatment of opioid use disorder and for the treatment of pain. Esmethadone is currently undergoing phase 3 clinical trials for the therapy of major depressive disorder (MDD). The results of a Phase 2 trial of esmethadone for MDD have been recently published, indicating promising efficacy, and confirming the absence of characteristic opiate effects or withdrawal after the end of treatment [Fava M, Stahl S, Pani L, De Martin S, Pappagallo M, Guidetti C. Alimonti A. Bettini E, Mangano R M, Wessel T, de Somer M, Caron J, Vitolo O V, DiGuglielmo G R, Gilbert A, Mehta H, Kearney M, Mattarei A, Gentilucci M, Folli F. Traversa S, Inturrisi C E, Manfredi P L, REL-1017 (Esmethadone) as Adjunctive Treatment in Patients With Major Depressive Disorder: A Phase 2a Randomized Double-Blind Trial. Am J Psychiatry. 2022; 179(2): 122-131. doi: 10.1176/appi.ajp.2021.21020197]. Although universally considered as a useful alternative to other opioids, a number of unexpected deaths have been associated with the use of racemic methadone, potentially due to a prolongation of the corrected QT interval (QTc) on the electrocardiogram (ECG).

The half-life of racemic methadone and its isomers, approximately 24-36 hours, suggests to those skilled in the art that a modified release formulation is unnecessary. In fact, for opioid use disorder, immediate release racemic methadone and levomethadone are administered once daily and, for the investigational uses of esmethadone for MDD and other neuropsychiatric disorders, immediate release esmethadone is also administered once daily. For pain, methadone and levomethadone can be administered daily but are generally administered more frequently, due to end of dose failure to provide effective analgesia for 24 hours.

### BRIEF SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

As described above, to date, modified release formulations (e.g., extended release formulations) of methadone and its isomers have not been produced or used, due to the perceived drawbacks of same, and due to the conventional wisdom that such formulations are not needed due to the long half-life of racemic methadone and its isomers. This long half-life of 24-36 hours appears ideal to those skilled in the art for a once-daily preparation. However, in contrast with the general assumption that a modified release formulation of methadone and its isomers is not needed because of its long half-life, the present inventors, who have been studying the PK and PD of methadone and its enantiomers for several decades, have come to the unexpected conclusion that a modified release formulation may decrease the potential for meaningful side effects at Cₘₐₓ and improve the therapeutic efficacy at Cₘᵢₙ (C_{trough}) concentration, despite the long half-life of racemic methadone and its isomers. This consideration is based on novel pharmacokinetic and pharmacodynamic knowledge acquired by the inventors, including novel pharmacokinetic and pharmacodynamic findings for esmethadone.

In the light of these considerations, an extended-release formulation of methadone, levomethadone or esmethadone could allow for safer and more effective therapies, characterized by improved stability in therapeutic plasma levels and potentially reduced inter-individual variability.

The present inventors, due to their combined experience with the use of methadone and its isomers, have come to the conclusion that for subgroups of patients, a formulation with a lower Cₘₐₓ, a longer Tₘₐₓ and a higher Cₘᵢₙ (C_{trough}) may have meaningful safety and efficacy advantages, including less side effects at peak concentrations, such as less euphoria, less sedation, less respiratory depression (racemic methadone and levomethadone), and less QTc prolongation (racemic methadone, levomethadone, and esmethadone), and less end of dose (Cₘᵢₙ, C_{trough}) therapeutic failure (racemic methadone, levomethadone, and esmethadone).

And so, certain aspects of the present invention provide a modified-release formulation or formulations for the delivery of racemic methadone, levomethadone or esmethadone. These modified release formulations may be oral, but can be formulations adapted to be delivered via other routes of administration—such as transdermal or transmucosal formulations or depot injectable formulations. These modified release oral formulations include oral modified release preparations that achieve modifications of pharmacokinetic (PK) parameters resulting in at least 10% lower Cₘₐₓ, at least 10% delayed Tₘₐₓ and/or at least 10% higher Cₘᵢₙ (C_{trough}) compared to immediate release formulations. In another embodiment, the modified-release formulation has a difference in Cₘₐₓ, Tₘₐₓ and Cₘᵢₙ that is at least 20% lower (Cₘₐₓ), 20% longer (Tₘₐₓ) or 20% higher Cₘᵢₙ) compared to immediate release formulations. In one embodiment, these modified release oral formulations are hydrogel preparations. In another embodiment, these modified-release preparations also include transdermal preparation with similarly advantageous pharmacokinetic profiles: at least 10% lower Cₘₐₓ, at least 10% longer Tₘₐₓ and or at least 10% higher Cₘᵢₙ compared to currently available oral immediate release formulations.

Additionally, it is understood that each patient differs in terms of drug disposition after administration. And so, another aspect of the present invention includes first obtaining the Cₘₐₓ, Tₘₐₓ, and Cₘᵢₙ for an oral immediate release formulation (of racemic methadone, Jevomethadone, or esmethadone) in a patient, and then administering an oral modified release formulation that results in at least 10% lower Cₘₐₓ, at least 10% delayed Tₘₐₓ and/or at least 10% higher Cₘᵢₙ (C_{trough}) compared to the Cₘₐₓ, Tₘₐₓ, and Cₘᵢₙ that was obtained for the oral immediate release formulation. Another embodiment of this aspect includes first obtaining the Cₘₐₓ, Tₘₐₓ, and Cₘᵢₙ for an oral immediate release formulation (of racemic methadone, levomethadone, or esmethadone) in a patient, and then administering a transdermal controlled release formulation that results in at least 10% lower Cₘₐₓ, at least 10% delayed Tₘₐₓ and/or at least 10% higher Cₘᵢₙ (C_{trough}) compared to the Cₘₐₓ, Tₘₐₓ, and Cₘᵢₙ that was obtained for the oral immediate release formulation. In another embodiment the transdermal formulation results in at least a 20% lower Cₘₐₓ, at least 10% delayed Tₘₐₓ and/or at least 10% higher Cₘᵢₙ (C_{trough}) compared to the Cₘₐₓ, Tₘₐₓ, and Cₘᵢₙ that was obtained for an oral immediate release formulation.

Additionally, in certain embodiments, the dissolution rate in vitro of the formulation, when measured by standard USP Drug Release test of U.S. Pharmacopeia XXVI (2003), is less than about 50% within about 6 hours and at least about 90% within about 24 hours, and having a maximum rate of release from about 5% to about 20% per hour, (and in another embodiment having a maximum rate of release from about 8% to about 10% per hour). In certain embodiments, the release rate in vitro is less than about 20% within about 8 hours and at least about 90% within about 24 hours, and having a maximum rate of release from about 5% to about 20% per hour, (and in another embodiment having a maximum rate of release from about 8% to about 12% per hour).

Other aspects of the present invention provide an oral dosage form comprising a racemic methadone, levomethadone, or esmethadone, or one or more salts thereof, in modified-release form. which, at steady state, provides an in vivo plasma profile of a maximum plasma concentration (Cₘₐₓ) at about 3-6 hours after administration or a Tₘₐₓ about 10% longer than the immediate release formulation.

Hence, a multiplicity of factors interplays in complex ways to determine the potential advantages associated with modified release formulations for methadone and its isomers, based on certain specific modifications to certain specific pharmacokinetic parameters. As stated, the specific modifications to these specific pharmacokinetic parameters. irrespective from the administration route and the formulation, can be quantified as at least 10% lower Cₘₐₓ, at least 10% higher Tₘₐₓ and/or at least 10% higher Cₘᵢₙ compared to immediate release formulations. The factors playing a pivotal role in this interplay, include deep knowledge of potential patient population and indications (see also previous applications by the inventors for racemic methadone, its isomers, its metabolites and its derivatives) and also include the physical, structural, pharmacokinetic, pharmacodynamic characteristics of the drugs disclosed in these novel formulations, including but not limited to:
Liposolubility and hydrosolubility
Partition coefficient
Structure
Structure activity relationship
Intestinal transporters, including P glycoprotein
BBB transporters, including P glycoprotein
Metabolism including CYP450 enzymes, including victim and offender actions, including specific enzymatic pathways of relevance such as CYP4502B6 and others of relevance
Methadone metabolites, including EDDP and EMDP
Binding to plasma proteins including binding to alfa-1-glycoprotein and the effects of inflammation on levels of alfa-1-glycoprotein in select patients
Binding to tissues, including CNS and tissues outside of the CNS
Binding to different receptors
Activity at different receptors
Block of different channels
Loading dose as a mean to improve efficacy and or obtain rapid efficacy
Breakthrough dose as a mean to improve efficacy
Differential effects and differential tolerance to these effects
Safety tolerability and implications for different indications
Efficacy and implication for different indications: loading dose and end of dose failure
QTc effects and relation of QTc effects to Cₘₐₓ and arrhythmias
Individual predisposition to cardiac risks from QTc prolonging drugs
Individual predisposition to opioid use disorder

The inventors have studied and disclosed their discoveries about a multiplicity of characteristics of methadone and its isomers, including those listed above. Taken together, this body of knowledge has induced the inventors to disclose that modified release preparations of these molecules, designed to achieve specific modifications of the immediate release formulations in current clinical use, may have clinically meaningful safety, tolerability and efficacy advantages over immediate release preparations. These advantages are not apparent to those skilled in the art mainly because the long half-life of methadone and its isomers suggests to those skilled in the art that a modified release formulation would not meaningfully improve safety, tolerability and efficacy. In particular those skilled in the art are of the general opinion that lowering Cₘₐₓ, prolonging Tₘₐₓ and increasing Cₘᵢₙ (C_{trough}) of methadone, levomethadone and esmethadone would not result in a meaningful benefit because of the long half-life of these drugs and therefore these select modified release preparations of these drugs, resulting in at least 10% lower Cₘₐₓ, and or at least 10% longer Tₘₐₓ, and or at least 10% higher Cₘᵢₙ (C_{trough}), compared to immediate release formulations have not been previously disclosed or pursued. The present inventors base their claim that preparations of these drugs, resulting in at least 10% lower Cₘₐₓ, and or at least 10% longer Tₘₐₓ, and or at least 10% higher Cₘᵢₙ (C_{trough}), compared to immediate oral release formulations may result in meaningful benefit for select patients with a medical need for methadone or its isomers who are at high risk for Cₘₐₓ induced side effects, including cardiac risks, or are at high risk for Cₘᵢₙ (C_{trough}) induced end of dose failure, including relapse in substance use disorder or depression. Therapeutic failure or side effects of immediate release formulations may also be due to fluctuations in drug levels outside of therapeutic window delimitated by tolerated Cₘₐₓ and effective Cₘᵢₙ.

Based on their joint body of knowledge on methadone and its isomers, and their derivatives, the inventors have determined that 10% lower Cₘₐₓ, and or 10% longer Tₘₐₓ, and or 10% higher Cₘᵢₙ (C_{trough}), may determine clinically meaningful improvements in safety, tolerability and efficacy compared to immediate release formulations. The inventors disclose that modified release formulations achieving 9% lower Cₘₐₓ or less, and 9% longer Tₘₐₓ or less, and 9% higher Cₘᵢₙ (C_{trough}) or less, compared to immediate release formulations, are unlikely to have a clinically meaningful impact. Based on the above determinations the inventors disclose the formulations and uses thereof described below.

Unexpected deaths have been associated with the use of racemic methadone, alone or more often with concomitant drugs. Opioid receptor related respiratory depression has generally been the presumed causing effect of these death, however cardiac arrhythmias from drug-induced QTc prolongation have been implicated by the present inventors and other authors. The QT interval describes the duration of the electric ventricular systole (i.e., the contraction of the myocardium of the left and right ventricles), which is measured from the beginning of the QRS complex to the end of the T wave. Prolongation of cardiac repolarization results in a lengthened QT interval on the surface ECG. Since the QT interval varies with heart rate (decreases at high heart rates and increases at low heart rates), it must be corrected for heart rate, by calculating the corrected QT interval or QTc. After the first observations on the QTc prolonging effects of methadone, recent evidence confirmed that methadone administration is associated with a QTc prolongation and cases of a life-threatening ventricular arrhythmia called torsades de pointes (TdP). This specific form of polymorphic ventricular tachycardia occurs in patients with a prolongation of the QT interval, either congenital or drug-induced. It is characterized by rapid and irregular QRS complexes, which appear to be twisting around the ECG baseline. QT-interval prolongation predisposes to the arrhythmia by prolonging repolarization, and this arrhythmia may either cease spontaneously or degenerate into ventricular fibrillation. In the latter case, it causes significant hemodynamic compromise and often death. For this reason, methadone carries a black box warning about this risk (methadone label).

Interestingly, as noted by the present inventors, drug induced QTc prolongation from methadone and its isomers are dose dependent. Thus, the risk of arrhythmias from methadone-induced QTc prolongation is dose dependent. Side effects for esmethadone, including nausea and vomiting are dose dependent and concentration dependent [Bernstein G, Davis K, Mills C, Wang L, McDonnell M, Oldenhof J, Inturrisi C, Manfredi PL, Vitolo O V. Characterization of the Safety and Pharmacokinetic Profile of D-Methadone, a Novel N-Methyl-D-Aspartate Receptor Antagonist in Healthy, Opioid-Naive Subjects: Results of Two Phase 1 Studies. J Clin Psychopharmacol. 2019 May/Jun;39(3):226-237]. Side effects for racemic methadone and levomethadone are dose dependent and concentration dependent, as illustrated in the methadone label and levomethadone label. Opioid agonist side effects from racemic methadone, including respiratory depression are due to the levo-isomer, levomethadone (Pasternak G W, Pan Y X. Mu opioids and their receptors: evolution of a concept. Pharmacol Rev. 2013;65(4):1257-1317. Published 2013 Sep 27. doi:10.1124/pr.112.007138; Drug Enforcement Administration (DEA). Diversion Control Division. Drug & Chemical Evaluation Section. Methadone. December 2019. https://www.deadiversion.usdoj.gov/drug_chem_info/methadone/methadone.pdf#search=methadone). The present inventors have noted that the side effects, including QTc prolongation, of esmethadone and thus levomethadone and racemic methadone, are not only dose dependent but Cₘₐₓ dependent, and, therefore, could potentially be reduced with a modified release formulation that could potentially reduce Cₘₐₓ while maintaining therapeutic drug levels.

It should be noticed that Cₘₐₓ and Tₘₐₓ are inversely correlated, and a modified-release formulation with a longer Tₘₐₓ will have a lower Cₘₐₓ and less risk for causing drug-induced QTc prolongation compared to an immediate release formulation at the same dose. Racemic methadone, levomethadone and esmethadone are presently formulated as an immediate release BCS Class I formulations. Therefore, a modified release formulation with a longer Tₘₐₓ compared to the immediate release BCS Class I formulation potentially results in a lower Cₘₐₓ, reducing the risk of QTc prolongation and TdP, compared to the immediate release formulation. This may be especially relevant in a subset of patients at risk for arrhythmias, as for example patients with mutations in the human ether-à-go-go-related gene (hERG). The hERG gene codes for channels mediating the ‘rapid’ delayed rectifier potassium current playing an important role in ventricular repolarization. The pharmacological inhibition of native IKr and hERG channels is a shared feature of diverse drugs associated with TdP. Select patients may be more susceptible to the sedative or respiratory depressant actions of racemic methadone or levomethadone, or to the euphoric effects of racemic methadone or levomethadone. A subset of patients may suffer from Cₘₐₓ related side effects, e.g., nausea and vomiting after the initial 75 mg loading dose of esmethadone used in clinical trials. Nausea and vomiting after the 75 mg dose could lead to decreased serum levels (similar to serum levels obtained with the 50 mg dose (Bernstein 2019). This potential drawback of the immediate release formulation at 75 mg is disclosed in the present application for the first time. A modified-release formulation with a lower Cₘₐₓ and a longer Tₘₐₓ compared to the immediate release BCS Class I formulation will then result in less side effects, including less nausea and vomiting. Finally, a subset of patients may be susceptible to end of dose therapeutic failure secondary to a low Cₘᵢₙ (C_{trough}). For this subset of patients, a modified release formulation with higher Cₘᵢₙ compared to the immediate release BCS Class I formulation will result in improved efficacy.

Studies performed by the present inventors demonstrate that esmethadone is a BCS Class I drug (see Example 1, below, with complete information/data supporting the high solubility, high permeability, gastric stability, and rapid dissolution of esmethadone proposed drug substance/drug product as per the FDA's BCS Guidance). Based on physicochemical and structural similarities among isomers, the present inventors conclude that the same is applicable for levomethadone and thus for racemic methadone. Studies performed by the present inventors shows block of potassium currents for esmethadone, levomethadone), and racemic methadone and the main metabolite EDDP (see Example 2, below, detailing manual patch electrophysiological studies). Studies demonstrate Cₘₐₓ dependent esmethadone (and levomethadone) QTc prolongation. Based on physicochemical and structural similarities among isomers, the present inventors conclude that the same is applicable for levomethadone and thus for racemic methadone (see Example 3, below, showing correlation of QTc with Cₘₐₓ).

A modified release drug formulation of esmethadone resulting in a Tₘₐₓ of at least 10% longer compared to the immediate release BCS Class I formulation of the same drug will have a lower Cₘₐₓ and less QTc prolonging effect, thereby potentially lowering the risk of arrhythmias in a meaningful way.

A modified release drug formulation of esmethadone resulting in a Tₘₐₓ of at least 10% longer compared to the immediate release BCS Class I formulation of the same drug will have less QTc prolonging effect, thereby lowering the risk of arrhythmias.

A modified release drug formulation of esmethadone resulting in a Tₘₐₓ of at least 10% longer compared to the immediate release BCS Class I formulation of the same drug will have less a lower Cₘₐₓ and will be less likely to induce Cₘₐₓ dependent side effects such as nausea and vomiting and other side effects.

A modified release drug formulation of a esmethadone resulting in a Cₘᵢₙ of at least 10% higher compared to the BCS Class I formulation of the same drug will have less probability of end of dose therapeutic failure.

A subset of subjects is at higher risk for drug induced QTc prolongation because of genetic reasons (hERG mutations). These genetically predisposed subjects may also be exposed to QTc prolonging environmental agents, including concomitant drugs and even food effects (a high carbohydrate load may determine additional QTc prolongation adding to the risk). This risk of arrhythmias is particularly meaningful in subjects when higher doses of QTc prolonging drugs are used (e.g., at the time of a loading dose or when higher doses are needed). While esmethadone is generally expected to be used at relatively low doses (once daily oral 25 mg tablet), a loading dose of 75 mg has been shown to result in rapid antidepressant effects in patients with major MDD (Fava 2022). Patients with MDD are at risk for suicide and therefore rapid onset effective antidepressant effects are desirable to lower the risk of suicide. On this therapeutic regimen, the highest Cₘₐₓ and therefore the highest risk of QTc prolongation and arrhythmias is expected on the first day of treatment approximately 150 minutes after the 75 mg loading dose. Some degree of QTc prolongation is also expected on subsequent days, and the peak for this QTc prolongation is seen approximately 150 minutes after each daily dose, at Cmax. While for the great majority of patients who are at average risk of cardiac arrythmias a modified release formulation of methadone or its isomers is not warranted, subsets of patients identified via EKG or medical history may benefit from the decreased risk from a lower Cₘₐₓ of these drugs.

Additionally, a higher Cₘᵢₙ (trough level) may be desirable in order not to lose efficacy. While the impact of end of dose failure for esmethadone for MDD or other indications is not known, a more balanced peak-trough profile (AUC with lower Cₘₐₓ and higher Cₘᵢₙ) may be desirable to improve or maintain efficacy.

While, for MDD, 25 mg may be an adequate therapeutic dose as suggested by recent evidence (Fava et al. 2022), esmethadone may be useful for a multiplicity of other diseases and disorders, as previously disclosed by the inventors in applications different related applications cited in the disclosure. For these diseases and disorders different esmethadone dosages, including dosages higher than 25 mg daily or higher than the 75 mg loading dose, may be needed. When higher doses are administered, Cₘₐₓ is higher and QTc prolonging effects may acquire increasing clinically relevance, making a modified-release formulation safer compared to an immediate release formulation, in particular for a subset of patients at higher risk for QTc associated arrhythmias. Additionally, when higher doses are administered, Cₘₐₓ is higher and the probability of Cₘₐₓ dependent side effects, such as nausea and vomiting is increased, making a modified release formulation not only safer but more effective (nausea and vomiting may reduce absorption and impede the reaching of therapeutic serum levels) compared to an immediate release formulation, in particular for a subset of patients who are more prone to side effects.

A small subset of patients, likely less than 0.1% of the general population, may be at increased risk for drug-induced QTc prolongation related arrhythmias. For these patients, treatment with modified release racemic methadone, levomethadone and esmethadone may be preferable.

The present inventors calculated the decrease in QTc based on decrease in Cₘₐₓ of at least 1 ms per point percentage decrease.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 is a graph showing change-from-baseline QTcF (ΔQTcF) across time points with statistical modeling (Cardiodynamic population). LS mean and 90% CI are based on a linear mixed-effects model: ΔQTcF=Time+Treatment+Treatment*Time+Baseline QTcF. An unstructured covariance structure was used to specify the repeated measures (time within patient).

FIG. 2 is a graph showing placebo-corrected change-from-baseline QTcF (ΔΔQTcF) at each time point with statistical modeling (Cardiodynamic population). LS mean and 90% CI are based on a linear mixed-effects model: ΔQTcF=Time+Treatment+Treatment*Time+Baseline QTcF. An unstructured covariance structure was used to specify the repeated measures (time within patient).

FIG. 3 is a graph showing a plot of geometric mean esmethadone plasma concentrations over time (C-QTc population). FIG. 3 displays mean and ±SD from descriptive statistics. If mean—SD was below 0, 0 was substituted as concentrations below 0 are not biologically plausible.

FIG. 4 is a graph showing a scatter plot of QTcF versus RR by treatment (Cardiodynamic population). The solid black line denotes the simple linear regression across all pairs of (RR,QTc). This line is based on the equation: QTcF=379.87+0.030×RR with a P value of <0.0001 for the slope.

FIG. 5 is a graph showing a QTcF-RR decile plot by treatment (Cardiodynamic population). The solid lines with dark gray and light gray shaded areas denote the 90% CI from the linear mixed effect model QTcF=intercept+slope×RR. The circles and squares with vertical bars denote the observed mean QTcF with 90% CI displayed at the median RR within each decile for placebo and esmethadone. respectively. The slope for placebo was 0.0063 with a P value of 0.2993. The slope for esmethadone was 0.018 with a P value of <0.0001.

FIG. 6 is a graph showing a scatter plot of observed esmethadone plasma concentrations and ΔQTcF by patient (C-QTc population). The light gray line and the dark gray shaded area denotes the LOESS regression and 90% confidence limits. The black solid line denotes the simple linear regression line. The plotted points denote the pairs of observed esmethadone plasma concentrations and ΔQTcF.

FIG. 7 is a graph showing a predicted ΔΔQTcF interval at geometric mean peak esmethadone concentrations for the model with esmethadone alone (C-QTc population). The solid black line with gray shaded area denotes the model-predicted mean ΔΔQTcF with 90% CI, which is calculated from the equation ΔΔQTcF (ms)=3.11 (ms)+0.014 (ms per ng/mL)×esmethadone plasma concentration (ng/mL). The whiskers (i.e., the vertical lines extending above and below the square and the triangle) denote the estimated mean (90% CI) ΔΔQTcF with plotted points at the geometric mean Cₘₐₓ of esmethadone.

FIG. 8 is a graph showing log transformation model predicted ΔQTcF and observed ΔQTcF across deciles of esmethadone plasma concentrations. The analysis is based on PK/QTc population. Squares with vertical bars denote the observed mean ΔΔQTcF with 90% CI displayed at the median plasma concentration within each decile. The solid black line with gray shaded area denotes the model-predicted mean ΔΔQTcF with 90% CI. The horizontal light gray line with notches shows the range of concentrations divided into deciles for esmethadone.

FIG. 9 is a graph showing a linear model (excluding outliers) predicted ΔQTcF and observed ΔQTcF across deciles of esmethadone plasma concentrations. The analysis is based on PK/QTc population. Squares with vertical bars denote the observed mean ΔΔQTcF with 90% CI displayed at the median plasma concentration within each decile. The solid black line with gray shaded area denotes the model-predicted mean ΔΔQTcF with 90% CI. The horizontal light gray line with notches shows the range of concentrations divided into deciles for esmethadone.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described above, to date, modified release formulations (e.g., extended release formulations) of methadone and its isomers have not been produced or used, due to the perceived drawbacks of same, and to the conventional wisdom that such formulations are not needed due to the lengthy half-life of racemic methadone and its isomers. However, in contrast with the general assumption that a modified-release formulation of methadone and its isomers is not needed because of its long half-life, the present inventors, who have been studying the PK and PD of methadone and its enantiomers for several decades, have come to the unexpected conclusion that a modified release formulation may decrease the potential for side effects at Cₘₐₓ and improve the therapeutic efficacy at Cₘᵢₙ (C_{trough}) concentration.

In the light of these considerations, an extended-release formulation of methadone, levomethadone or esmethadone could allow for safer and more effective therapies, characterized by improved stability in therapeutic plasma levels and potentially reduced inter-individual variability.

The present inventors, due to their combined experience with the use of methadone and its isomers, have come to the conclusion that for subgroups of patients, a formulation with a lower Cₘₐₓ, a longer Tₘₐₓ and a higher Cₘᵢₙ (C_{trough}) may have important safety and efficacy advantages, including less side effects at peak concentrations, such as less euphoria, less sedation, less respiratory depression (racemic methadone and levomethadone), and less QTc prolongation (racemic methadone, levomethadone, and esmethadone), and less end of dose (Cₘᵢₙ, C_{trough}) therapeutic failure (racemic methadone, levomethadone, and esmethadone).

And so, certain aspects of the present invention provide a modified-release formulation of formulations for the delivery of racemic methadone, levomethadone or esmethadone. These modified release formulations may be oral, but can be formulations adapted to be delivered via other routes of administration—such as transdermal or transmucosal formulations. These modified release oral formulations include oral modified release preparations that achieve modifications of pharmacokinetic (PK) parameters resulting in at least 10% lower Cₘₐₓ, at least 10% delayed Tₘₐₓ and/or at least 10% higher Cₘᵢₙ (C_{trough}) compared to immediate release formulations. In another embodiment, the release formulation has a difference in Cₘₐₓ, Tₘₐₓ and Cₘᵢₙ that is at least 20% lower (Cₘₐₓ), 20% longer (Tₘₐₓ) or 20% higher Cₘᵢₙ) compared to immediate release formulations. In one embodiment, these modified release oral formulations are hydrogel preparations. In another embodiment, these modified release preparations also include transdermal preparation with similarly advantageous pharmacokinetic profiles: at least 10% lower Cₘₐₓ, at least 10% longer Tₘₐₓ and or at least 10% higher Cₘᵢₙ compared to oral immediate release formulations (BCS-1 formulations).

Additionally, it is understood that each patient differs in terms of drug disposition after administration. And so, another aspect of the present invention includes first obtaining the Cₘₐₓ, Tₘₐₓ, and Cₘᵢₙ for an oral immediate release formulation (of racemic methadone, levomethadone, or esmethadone) in a patient, and then administering an oral modified release formulation that results in at least 10% lower Cₘₐₓ, at least 10% delayed Tₘₐₓ and/or at least 10% higher Cₘᵢₙ (C_{trough}) compared to the Cₘₐₓ, Tₘₐₓ, and Cₘᵢₙ that was obtained for the oral immediate release formulation. Another embodiment of this aspect includes first obtaining the Cₘₐₓ, Tₘₐₓ, and Cₘᵢₙ for a oral immediate release formulation (of racemic methadone, levomethadone, or esmethadone) in a patient, and then administering a transdermal controlled release formulation that results in at least 10% lower Cₘₐₓ, at least 10% delayed Tₘₐₓ and/or at least 10% higher Cₘᵢₙ (C_{trough}) compared to the Cₘₐₓ, Tₘₐₓ, and Cₘᵢₙ that was obtained for the oral immediate release formulation.

Additionally, in certain embodiments, the dissolution rate in vitro of the formulation, when measured by standard USP Drug Release test of U.S. Pharmacopeia XXVI (2003), is less than about 50% within about 6 hours and at least about 90% within about 24 hours, and having a maximum rate of release from about 5% to about 20% per hour, (in another embodiment having a maximum rate of release from about 8% to about 10% per hour). In certain embodiments, the release rate in vitro is preferably less than about 20% within about 8 hours and at least about 90% within about 24 hours, and having a maximum rate of release from about 5% to about 20% per hour, (in another embodiment having a maximum rate of release from about 8% to about 12% per hour).

Other aspects of the present invention provide an oral dosage form comprising a racemic methadone, levomethadone, or esmethadone, or one or more salts thereof, in modified-release form, which, at steady state, provides an in vivo plasma profile of a maximum plasma concentration (Cₘₐₓ) at about 3-6 hours after administration or a Tₘₐₓ about 10% longer than the immediate release formulation.

Oral dosage forms with 0 to 12 hours release profiles are known in the art, as are oral dosage forms with 12-24-hour release profiles. It is a common opinion that drugs like methadone, levomethadone or esmethadone do not need a modified controlled release dosage form owing to their long circulation time (long half-life). Modified-release dosage forms detailed in the current invention can provide substantially constant or therapeutically consistent levels of the above drugs, avoiding meaningful intensity peaks in effects, such as nausea, vomiting, or drowsiness, which are often associated with high blood levels of these drugs and the peak concentration Cₘₐₓ. It is the intent of the present modified-release formulation to reduce the intensity or degree of the above side effects. In certain embodiments, the dosage form is efficacious in a human in the fed or fast state.

For esmethadone, the linearity of PK was conclusively demonstrated for multiple-dose parameters (Bernstein 2019). These results are consistent with results reported following administration of racemic methadone (Foster D J R, Somogyi A A, White J M, et al. Population pharmacokinetics of (R)-, (S)- and rac-methadone in methadone maintenance patients. Br J Clin Pharmacol. 2004:57:742-755). Therefore, dose proportionality and linearity after oral administration of each of the three molecules, based on the data from Bernstein 2019 and Foster 2004 are expected. Therefore, the actual Cₘₐₓ after administration of 5 mg of immediate release esmethadone and the expected Cₘₐₓ after administration of 5 mg of levomethadone or racemic methadone is 53.3 ng/ml.

As described above, one aspect of the invention includes a modified release preparation of esmethadone, levomethadone, or racemic methadone that results in a Cₘₐₓ lower by at least 10% compared to the Cₘₐₓ reached with the corresponding immediate release preparation. And so, in the case of a 5 mg dose, the Cₘₐₓ for the modified release formulation is 48 ng/ml or less instead of the 53.3 ng/ml of the immediate release formulation.

In another example, in the case of a 20 mg dose, the Cₘₐₓ for the modified release formulation is 147 ng/ml or less instead of the 163.3 ng/ml of the immediate release formulation.

In another example. in the case of a 60 mg dose, the Cₘₐₓ, for the modified release formulation is 363 ng/ml or less instead of the 403.73 ng/ml of the immediate release formulation.

In another example, in the case of a 100 mg dose, the Cₘₐₓ for the modified release formulation is 664 ng/ml or less instead of the 738.7 ng/ml of the immediate release formulation.

In another example, in the case of a 150 mg dose, the Cₘₐₓ for the modified release formulation is 952 ng/ml or less instead of the 1057 ng/ml of the immediate release formulation.

In another example, in the case of a 200 mg dose, the Cₘₐₓ for the modified release formulation is 1377 ng/ml or less instead of the 1530 ng/ml of the immediate release formulation.

As described above, in another embodiment, the modified release preparation of esmethadone, levomethadone or racemic methadone results in a Cₘₐₓ lower by at least 20% compared to the Cₘₐₓ reached with the corresponding immediate release preparation. And so, in the case of a 5 mg dose, the Cₘₐₓ for the modified release formulation is 42.6 ng/ml or less instead of the 53.3 ng/ml described above for the immediate release formulation.

And so, in the case of a 20 mg dose, the Cₘₐₓ for the modified release formulation is 130.6 ng/ml or less instead of the 163.3 ng/ml of the immediate release formulation.

In another example, in the case of a 60 mg dose, the Cam for the modified release formulation is 323 ng/ml or less instead of the 403.7 ng/ml of the immediate release formulation.

In another example, in the case of a 100 mg dose, the Cₘₐₓ for the modified release formulation is 591 ng/ml or less instead of the 738.7 ng/ml of the immediate release formulation.

In another example, in the case of a 150 mg dose, the Cₘₐₓ for the modified release formulation is 845.6 ng/ml or less instead of the 1057 ng/ml of the immediate release formulation.

In another example, in the case of a 200 mg dose, the Cₘₐₓ for the modified release formulation is 1224 ng/ml or less instead of the 1530 ng/ml of the immediate release formulation.

Another aspect of the invention includes a modified release preparation of esmethadone, levomethadone or racemic methadone that results in a Tₘₐₓ at least 10% longer compared to the Tₘₐₓ reached with the corresponding immediate release preparation. In the case of a 5 mg dose the Tₘₐₓ for the modified release formulation is at least 165 minutes or longer instead of the 150 minutes of the immediate release formulation.

In another embodiment, the modified release preparation of esmethadone, levomethadone or racemic methadone results in a Tₘₐₓ at least 20% longer compared to the Tₘₐₓ reached with the corresponding immediate release preparation. Thus, in the case of a 5 mg dose, the Tₘₐₓ for the modified release formulation is at least 180 minutes or longer instead of the 150 minutes of the immediate release formulation.

Additionally, the Cₗₐₛₜ of d-methadone for 25 mg, 50 mg and 75 mg of the PK Population defined in Bernstein et al. 2019 measured at Day 1 after the first administration, were 43.23, 87.60, and 108.3 ng/ml, respectively. Steady state Curough concentrations (measured at Day 10) were 142.7, 326.2 and 338.8 ng/ml respectively.

Therefore, the actual Cₘᵢₙ after administration of 25 mg, 50 mg and 75 mg of immediate release esmethadone and the expected Cₘᵢₙ after administration of 25 mg, 50 mg and 75 mg of levomethadone or 25 mg, 50 mg or 75 mg of racemic methadone are 43.23, 87.60, and 108.3 ng/ml.

While a 10% difference may be clinically meaningful, e.g., for select patients at high risk for fatal arrhythmias caused by QTc prolongation by QTc prolonging drugs, an alternate modified release formulation has a difference in Cₘₐₓ, Tₘₐₓ and Cₘᵢₙ that is at least 20% lower (Cₘₐₓ), longer (Tₘₐₓ) or higher Cₘᵢₙ) compared to immediate release formulations.

Therefore, the claimed Cₘᵢₙ after administration of 25 mg, 50 mg and 75 mg of modified release esmethadone and the claimed Cₘᵢₙ after administration of 25 mg, 50 mg and 75 mg of levomethadone and the claimed Cₘᵢₙ after administration of 25 mg, 50 mg or 75 mg of racemic methadone are 47.46, 96.36 and 119.1 ng/ml or, in another embodiment, 51.876, 105.12, 129.96 ng/ml, respectively. This modified release preparation will reduce end of dose therapeutic failure and will help obtain a faster achievement of the steady state concentration in a multiple dose regimen.

It is understood that each patient differs in terms of drug disposition after administration and the values reported above are means from study populations and therefore the individual values will be different from the mean values. However, the modified release preparation aims to determine for each individual patient a difference in Cₘₐₓ, Tₘₐₓ and Cₘᵢₙ that is at least 10% lower (Cₘₐₓ), longer (Tₘₐₓ) or higher Cₘᵢₙ) compared to immediate release preparations and aims to determine for each individual patient a difference in Cₘₐₓ, Tₘₐₓ and Cₘᵢₙ that is at least 20% lower (Cₘₐₓ), longer (Tₘₐₓ) or higher Cₘᵢₙ) compared to immediate release preparations. The modified or controlled release formulations of racemic methadone, levomethadone and esmethadone may be oral but can also be transdermal or depot preparations.

“Oral dosage form” as used herein means a unit dosage form prescribed or intended for oral administration.

“Modified-release” as used herein means to include the release of the drug (i.e., methadone, levomethadone or esmethadone) at such a rate that blood levels are maintained within a therapeutic range but below toxic levels for at least about 24 hours after administration at steady state. The term “controlled release” can be used interchangeably. The term “steady state” means that an equilibrium plasma level for a given drug has been achieved and is maintained with subsequent doses of the drug at a level at or above the minimum effective therapeutic level and below the minimum toxic plasma level for a given drug i.e., inside the therapeutic window of the drug. For example, the target therapeutic plasma levomethadone concentration for the treatment of opioid use disorder in methadone maintenance program (MMT) described in Mannaioni et al. was set at 80-250 ng/ml (Mannaioni G. Lanzi C, Lotti M, et al. Methadone Dose Adjustments, Plasma R-Methadone Levels and Therapeutic Outcome of Heroin Users: A Randomized Clinical Trial. Eur Addict Res. 2018:24(1):9-18. doi: 10.1159/000485029). It is understood by those ordinarily skilled in the art that such symptoms and effects vary between individuals and that the outcome measurements are subjective.

“Cₘₐₓ” as used herein means the measured concentration of the drug in the plasma at the point of maximum concentration.

“Cₘᵢₙ” as used herein means the measured concentration of the drug in the plasma at the point of minimum concentration, just before the administration of the following dose in a multiple dose regimen.

“C₂₄” as used herein means the measured concentration of the drug in the plasma at about 24 hours.

“C₁₂” as used herein means the measured concentration of the drug in the plasma at about 12 bours.

“AUC” as used herein means the area under the curve measured from one time to another, the curve is obtained from the drug plasma concentrations over the time.

Pharmaceutically acceptable sales of methadone, levomethadone or esmethadone include, but are not limited to, metal salts, such as sodium salt, potassium salt, cesium salt and the like; alkaline earth metals, such as calcium salt, magnesium salt and the like; organic amine salts, such as triethylamine salt, pyridine salt, picoline salt, ethanolamine salt, triethanolamine salt, dicyclohexylamine salt. N,N′-dibenzylethylenediamine salt and the like; inorganic acid salts, such as hydrochloride, hydrobromide, sulfate, phosphate and the like: organic acid salts, such as formate, acetate, trifluoroacetate, maleate, tartrate and the like; sulfonates, such as methanesulfonate, benzenesulfonate. p-toluenesulfonate, and the like; and amino acid salts, such as arginate, asparginate, glutamate and the like.

The in vivo modified release can be, in some cases, correlated to a decreased dissolution rate of the dosage form that yields a modified drug absorption. Different dosage forms can be eventually compared in vitro by studying the dissolution rate in vitro. The maximum dissolution rate can be measured by assessing the slope of the dissolution profile when the active ingredient is 50% dissolved with respect to the half-hour before and half-hour after about 50% dissolution is achieved. Measuring the percentage of active ingredient dissolved can be done by spectroscopy techniques, as well as other well know techniques in the art. In certain embodiments of all oral dosage forms mentioned herein, the maximum dissolution rate is from about 3% to about 50% per hour, (in another embodiment from about 5% to about 40% per hour; in another embodiment from about 7% to about 30% per hour; and in yet another embodiment from about 8% to about 25% per hour). The dissolution rate in vitro is measured as described by standard Drug Release test of U.S. Pharmacopeia.

In still a further embodiment, the oral dosage form provides. at steady state, 10% lower Cₘₐₓ, and or 10% longer Tₘₐₓ, and or 10% higher Cₘᵢₙ (C_{trough}), may determine clinically meaningful improvements in safety, tolerability and efficacy compared to immediate release formulations. At the same time, modified release formulations achieving 9% lower Cₘₐₓ or less, and 9% longer Tₘₐₓ or less, and 9% higher Cₘᵢₙ (C_{trough}) or less, compared to immediate release formulations, are unlikely to have a clinically meaningful impact.

The methadone, levomethadone or esmethadone in a modified release form may be a particle of drug that is combined with a release-retarding material. The release-retarding material is destrably a material that permits release of the drug at a sustained rate in an aqueous medium. The release-retarding material can be selectively chosen so as to achieve, in combination with the other stated properties, a desired in vivo release rate.

In certain embodiments, an oral dosage form of the invention is formulated to provide for an increased duration of drug activities, allowing once-daily dosing. In general, a release-retarding material is used to provide the increased duration of therapeutic intended action. However, it should be appreciated that the dosage form of the invention can be provided for more frequent dosage regiments, e.g., twice-daily, thrice-daily; etc.

Release-retarding materials include, but are not limited to, acrylic polymers, celluloses, alkylcelluloses, shellac, zein, hydrogenated vegetable oil, hydrogenated castor oil, and combinations thereof.

In certain embodiments of the oral dosage forms discussed herein, the release-retarding material is a pharmaceutically acceptable acrylic polymer, including acrylic acid and methacrylic acid copolymers, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer and glycidyl methacrylate copolymers. In certain embodiments, the acrylic polymer comprises one or more ammonio methacrylate copolymers. Ammonio methacrylate copolymers are well-known in the art, and are described in National Formulary XXI (“NF XXI”) as fully polymerized copolymers of acrylic and methacrylie acid esters with a low content of quaternary ammonium groups.

In other embodiments, the release-retarding material is a cellulosic material (e.g., a cellulosic polymer), such as an alkyl cellulosic material, such as ethylcellulose. Those ordinarily skilled in the art will appreciate that other cellulosie polymers, including other alkyl cellulosie polymers, can be substituted for part or all of the ethylcellulose.

The release-retarding material can also include an erosion-promoting agent. Any suitable erosion promoting agent can be used. Examples of such erosion promoting agents include, but are not limited to, starches and gums; an agent that makes microporous lamina in the environment of use, such as polycarbonates comprised of linear polyesters of carbonic acid in which carbonate groups reoccur in the polymer chain; and/or a semi-permeable polymer.

In one embodiment, the release-modifying agent can also act to retard the release of the active. The agent may be an anionic alkyl salt, such as sodium lauryl sulfate, metal stearate, and combinations thereof.

In certain embodiments, methadone, levomethadone or esmethadone in modified release forms can include a plurality of substrates comprising the active ingredient, which substrates are coated with a modified release coating comprising a release-retarding material. In certain other embodiments, the methadone, levomethadone or esmethadone in sustained-release form can include a plurality of substrates comprising the analgesic in a sustained-release matrix. Combinations of the foregoing substrates, matrices and coatings are also contemplated to be within the scope of aspects of the present invention provided herein.

The modified release preparations of aspects of the present invention can be made in conjunction with any multiparticulate system, such as beads, ion-exchange resin beads, spheroids, microspheres, seeds, pellets, granules, and other multiparticalate systems in order to obtain a desired sustained-release of the methadone, levomethadone or esmethadone. The multiparticulate system can be presented in a capsule or in any other suitable unit dosage form. In certain embodiments, more than one multiparticulate system can be used, each exhibiting different characteristics, such as pH-dependence of release, time for release in various media (e.g., acid, base, simulated intestinal fluid), release in vivo, size and composition.

In certain embodiments of the oral dosage forms described herein, the release- retarding material is in the form of a coating comprising an aqueous dispersion of a hydrophobic polymer. The inclusion of an effective amount of a plasticizer in the aqueous dispersion of hydrophobic polymer will further improve the physical properties of the film. For example, because ethylcellulose has a relatively high glass transition temperature and does not form flexible films under normal coating conditions, it is necessary to plasticize the ethylcellulose before using it as a coating material. Generally, the amount of plasticizer included in a coating solution is based on the concentration of the film-former, e.g., most often from about 1 to about 50 percent by weight of the film-former. Concentrations of the plasticizer, however, can be determined by routine experimentation.

Examples of plasticizers for ethylcellulose and other celluloses include, but are not limited to, dibutyl sebacate, diethyl phthalate, dibutyl phthalate, triethyl citrate, tributyl citrate, and triacetin, although it is possible that other plasticizers (such as acetylated monoglycerides, phthalate esters, castor oil, etc.) can be used.

Examples of plasticizers for the acrylic polymers include, but are not limited to, citric acid esters such as triethyl citrate, tributyl citrate, dibutyl phthalate, and possibly polyethylene glycols, propylene glycol, diethyl phthalate, castor oil, and triacetin, although it is possible that other plasticizers (such as acetylated monoglycerides, phthalate esters, castor oil, etc.) can be used.

The modified release profile of drug release in the formulations of aspects of the present invention (either in vivo or in vitro) can be altered. for example, by using more than one release-retarding material, varying the thickness of the release-retarding material, changing the particular release-retarding material used, altering the relative amounts of release-retarding material, altering the manner in which the plasticizer is added (e.g., when the sustained- release coating is derived from an aqueous dispersion of hydrophobic polymer), by varying the amount of plasticizer relative to retardant material, by the inclusion of additional ingredients or excipients, by altering the method of manufacture; etc.

In addition, a modified release matrix also can contain suitable quantities of other materials. e.g., diluente, lubricants, binders, granulating aids, colorants, flavorants and glidants that are conventional in the pharmaceutical art.

As outlined before, racemic methadone and levomethadone are approved for the treatment of pain rf and for the treatment of opioid use disorder, due to the agonistic affect towards the μ opioid receptor. Esmethadone exerts antagonism at NMDARs, and is currently under investigation for the therapy of treatment-resistant major depression and other neuropsychiatric disorders.

Aspects of the present invention also provides a method comprising orally administering to a fed or unfed human on a once daily basis an oral modified release dosage form of aspects of the present invention, whereupon pain in the human is treated. The administration of the modified release dosage form is continued over the dosing interval of a unit dose to maintain an adequate pharmacodynamic response with the modified release dosage form. In certain embodiments, the adequate pharmacodynamic response will last between about 12 and about 24 hours, (in a particular embodiment, about 24 hours. The administration of the modified release unit dosage form is continued over the dosing interval of the unit dose to maintain the adequate pharmacodynamic response with the modified release dosage form. If necessary, the above steps are repeated until a determination of adequate pharmacodynamic response is obtained with the modified release unit dosage form.

According to the above method, a patient can be titrated with a modified release methadone, levomethadone or esmethadone dosage form. Subsequent maintenance therapy can be provided with the same modified release dosage form. In one embodiment of the invention, an oral sustained-release dosage form, as described herein, orally administered to treat pain in a human, further comprises at least one release-retarding agent. The oral dosage form may further comprise at least one release-retarding agent and at least one plasticizer, and optionally at least one release-modifying agent. Select patients may use the modified release formulation regularly and the immediate release formulation as needed.

### EXAMPLES

### Example 1—Studies of Equilibrium Solubility, Permeability, and Dissolution of Esmethadone

### 1. Equilibrium Solubility Study

An equilibrium solubility study was performed to evaluate solubility of esmethadone HCl over the pH range of 1.2-6.8 at 37° C.±1° C. Solubility was evaluated in triplicate at pH 1.2. pH 4.5, and pH 6.8; with pH 6.8 representative of the lowest solubility within this range.

Solubility was determined in buffer solutions as described in USP <1236> (incorporated by reference herein in its entirety). Buffers were prepared according to USP Buffer Solutions chapter, section 4.1 (incorporated by reference berein in its entirety). The esmethadone HCl drug substance lot used in this study was manufactured using the proposed commercial process and characterized before use. For each pH level, the final pH of each sample replicate was confirmed to be within ±0.1 pH unit of the initial buffer pH for each respective buffer. Stability of the drug substance in each buffer was confirmed to be ≤ 10% (no increase in impurities was observed) over the total time of the study. Results are shown below in Table 1.

TABLE 1
Equilibrium Solubility of Esmethadone HCl at 37° C.

| | Esmethadone HCl (mg/mL) | | |
|---|---|---|---|
| Replicate# | pH 1.2 | pH 4.5 | pH 6.8 |
| 1 | 62.1 | 67.0 | 3.2 |
| 2 | 60.0 | 66.7 | 3.2 |
| 3 | 54.2 | 66.8 | 3.1 |
| Mean | 58.8 | 66.9 | 3.2 |

### Conclusion:

75 mg esmethadone HCl in 250 mL has a concentration of 0.3 mg/mL esmethadone HCl which represents the minimum concentration at which solubility would need to be demonstrated in order to for esmethadone HCl to be classified as highly soluble for the purposes of determining the BCS classification. All the equilibrium solubility results (mg/mL) exceed this minimum concentration with the lowest solubility result of 3.1 mg/mL esmethadone HCl being observed at pH 6.8. Based on these results, esmethadone HCl can be classified as a highly soluble compound for the purposes of determining the BCS classification according to criteria provided in ICH M9.

### 2. Caco-2 Cell Permeability Assay Study

The permeability of esmethadone HCl was assessed using a validated in vitro Caco-2 permeability model. The model was validated in an apical to basolateral (A-B, pH 6.5/7.4) and basolateral to apical (B-A, pH 6.5/7.4) bidirectional assay using a set of 23 compounds. These compounds were selected as representative of the range of expected percent absorption from low to moderate to high as recommended in Annex I of the Guidance for Industry: M9 Biopharmaceutics Classification System-Based Biowaivers (May 2021). The relationship between the observed in vitro permeability coefficients as compared to the expected percent absorption for all compounds was used to classify the test compound esmethadone HCl into a low, moderate, or high permeability category.

Four reference compounds (colchicine, labetalol, propranolol, and ranitidine) were included when the assay was performed. These compounds were selected to cover the range of low to high permeability coefficient and to include at least one substrate for the P glycoprotein (Pgp) efflux transporter (colchicine). Based on historical data obtained in the experimental model, the mean permeability value (Papp) for each reference compound was expected to fall within the predetermined specifications.

In order to demonstrate the functional expression of the Pgp efflux transporter system an additional specification for validation of the bidirectional assay was that the Pgp substrate colchicine should have an efflux ratio >2, where the efflux ratio is defined as the Papp(BA)/Papp(AB) for assays run on the same day.

Fluorescein was used as the zero-permeability marker in all Caco-2 cell assays as a quality control for the integrity of the Caco-2 monolayer. Fluorescein is not specifically listed in Annex I of the Guidance for Industry: M9 Biopharmaceuties Classification System-Based Biowaivers (May 2021). This marker has been used historically in the permeability model and a large data set exists demonstrating that this marker is not permeable in Caco-2 cells. Well-defined criteria (known or readily available to those of ordinary skill in the art) have been established for the acceptance of fluorescein results to validate monolayer integrity.

Testing concentrations for the in vitro permeability assessment were determined at 1%, 10%, and 100% of the highest strength dissolved in 250 mL. Test concentrations at 1 ug/mL. 10 ug/mL, and 100 ugimL were used in the study.

Results:

As shown in Table 2, below, the test compound esmethadone HCl showed a mean A-B permeability coefficient of 16.3, 15.2 and 21.3 x 10-6 cm/s when tested at 1, 10 and 100 pg/mL, respectively. Esmethadone HCl at all concentrations is considered a high permeability compound as determined by comparison to the A-B validation data set. The observed BA/AB efflux ratios were no higher than 3.2, well below the efflux ratios of reference transporter substrates included in the validation run. (Data for the reference compounds are shown in Table 3, below.) Therefore, esmethadone HCl does not appear to be a substrate of efflux transporters, and the measured in vitro permeability is considered independent of the testing concentration. Mean recovery values varied between 51-74% in the assays. In this test system it is normal to observe some loss of compound due to adsorption to the cell monolayer or to the trans-well plate itself. Recovery is not typically a complication for in vitro permeability categorization anless recoveries are extremely low (<20%) and distinction is needed between low and moderate permeability.

TABLE 2
Esmethadone HCl Permeability Results

| Compound | Test | Permeability (10⁻⁶ cm/s) | | | | Percent Recovery (%) | | | |
|---|---|---|---|---|---|---|---|---|---|
| I.D. | Conc. | Rep. 1 | Rep. 2 | Rep. 3 | Mean (SD) | Rep. 1 | Rep. 2 | Rep. 3 | Mean (SD) |

A-B permeability (Caco-2, pH 6.5/7.4)

| | | | | | | | | | | |
|---|---|---|---|---|---|---|---|---|---|---|
| esmethadone | 1 | μg/mL | 15.57 | 14.98 | 18.35 | 16.3 (1.8) | 53 | 51 | 51 | 51 (0.9) |
| HCl | | | | | | | | | | |
| esmethadone | 10 | μg/mL | 18.43 | 12.60 | 14.45 | 15.2 (3.0) | 57 | 54 | 60 | 57 (3.4) |
| HCl | | | | | | | | | | |
| esmethadone | 100 | μg/mL | 20.96 | 22.54 | 20.41 | 21.3 (1.1) | 64 | 64 | 56 | 61 (4.3) |
| HCl | | | | | | | | | | |

B-A permeability (Caco-2, pH 6.5/7.4)

| | | | | | | | | | | |
|---|---|---|---|---|---|---|---|---|---|---|
| esmethadone | 1 | μg/mL | 29.43 | 29.27 | 27.05 | 28.6 (1.3) | 65 | 58 | 59 | 60 (3.6) |
| HCl | | | | | | | | | | |
| esmethadone | 10 | μg/mL | 36.95 | 38.94 | 46.41 | 40.8 (5.0) | 67 | 74 | 82 | 74 (7.6) |
| HCl | | | | | | | | | | |
| esmethadone | 100 | μg/mL | 50.22 | 45.15 | 53.82 | 49.7 (4.4) | 74 | 82 | 68 | 74 (7.1) |
| HCl | | | | | | | | | | |

TABLE 3
Reference Compound Permeability Results

| Compound | Test | Permeability (10⁻⁶ cm/s) | | | | Percent Recovery (%) | | | |
|---|---|---|---|---|---|---|---|---|---|
| I.D. | Conc. | Rep. 1 | Rep. 2 | Rep. 3 | Mean (SD) | Rep. 1 | Rep. 2 | Rep. 3 | Mean (SD) |

A-B permeability (Caco-2, pH 6.5/7.4)

| | | | | | | | | | | | |
|---|---|---|---|---|---|---|---|---|---|---|---|
| Colchicine | 10 μM | 0.31 | 0.23 | 0.32 | 0.3 | (0.0) | 88 | 79 | 78 | 81 | (5.5) |
| Labetalol | 10 μM | 20.17 | 19.79 | 18.71 | 19.6 | (0.8) | 118 | 116 | 116 | 117 | (0.8) |
| Propranolol | 10 μM | 45.43 | 40.35 | 36.94 | 40.9 | (4.3) | 106 | 99 | 106 | 104 | (3.9) |
| Ranitidine | 10 μM | 0.80 | 0.69 | 0.58 | 0.7 | (0.1) | 100 | 99 | 104 | 101 | (2.3) |

B-A permeability (Caco-2, pH 6.5/7.4)

| | | | | | | | | | | | |
|---|---|---|---|---|---|---|---|---|---|---|---|
| Colchicine | 10 μM | 7.69 | 7.51 | 6.58 | 7.3 | (0.6) | 88 | 94 | 91 | 91 | (2.8) |
| Labetalol | 10 μM | 48.69 | 49.38 | 49.86 | 49.3 | (0.6) | 106 | 106 | 107 | 106 | (0.6) |
| Propranolol | 10 μM | 48.79 | 48.06 | 45.79 | 47.5 | (1.6) | 106 | 115 | 102 | 108 | (6.7) |
| Ranitidine | 10 μM | 2.54 | 2.03 | 2.66 | 2.4 | (0.3) | 86 | 96 | 92 | 91 | (5.0) |

Drug Substance Stability in the Gastrointestinal Tract

To support the permeability study, solutions at a target concentration of 100 ug/mL esmethadone HCl in simulated gastric fluid and simulated intestinal fluid were evaluated for degradation after storage at 37° ° C.for 1 hour and 3 hours, respectively, to represent the expected period of contact of the drug substance with these fluids in vivo. Results are shown in Tables 4 and 5, below.

TABLE 4
Gastrointestinal Solution Stability of Esmethadone HCl at 37° C.

| | | Incubation | % Compound Remaining | | | | Half-Life | | | |
|---|---|---|---|---|---|---|---|---|---|---|
| Compound | Test | Time | (% remaining) | | | | (minutes) | | | |
| I.D. | Conc. | (minutes) | Rep. 1 | Rep. 2 | Rep. 3 | Mean | Rep. 1 | Rep. 2 | Rep. 3 | Mean |

Half-life (simulated gastric fluid)

| | | | | | | | | | | |
|---|---|---|---|---|---|---|---|---|---|---|
| d-methadone | 100 μg/mL | 0 | 100.0 | 100.0 | 100.0 | 100 | >60 | >60 | >60 | >60 |
| HCl | | | | | | | | | | |
| d-methadone | 100 μg/mL | 60 | 110.2 | 127.3 | 90.0 | 109 | | | | |
| HCl | | | | | | | | | | |

Half-life (simulated intestinal fluid)

| | | | | | | | | | | |
|---|---|---|---|---|---|---|---|---|---|---|
| d-methadone | 100 μg/mL | 0 | 100.0 | 100.0 | 100.0 | 100 | >180 | >180 | >180 | >180 |
| HCl | | | | | | | | | | |
| d-methadone | 100 μg/mL | 180 | 89.6 | 96.6 | 105.9 | 97 | | | | |
| HCl | | | | | | | | | | |

TABLE 5
Gastrointestinal Solution Stability of Reference Compounds at 37° C.

| Compound | Test | Half-Life (minutes) | | | |
|---|---|---|---|---|---|
| I.D. | Conc. | Rep. 1 | Rep. 2 | Rep. 3 | Mean |

Half-life (simulated gastric fluid)

| | | | | | |
|---|---|---|---|---|---|
| Erythromycin | 10 μM | 6.4 | 6.5 | 6.4 | 6 |
| Propanthaline | 10 μM | >60 | >60 | >60 | >60 |

Half-life (simulated intestinal fluid)

| | | | | | |
|---|---|---|---|---|---|
| Chlorambucil | 10 μM | 26.2 | 26.2 | 25.7 | 26 |
| Propanthaline | 10 μM | 253.1 | 251.2 | 227.0 | >180 |

Gastrointestinal solution stability was performed along with the Caco-2 cell permeability assay study.

### Conclusion

Based on the results of the Caco-2 cell permeability assay method, high permeability has been demonstrated for esmethadone HCl independent of the testing concentration and with confirmation that compound does not undergo active efflux. In addition, stability of esmethadone HCl in the gastrointestinal tract for a period that is representative of the in vivo contact of the drug substance with these fluids has been demonstrated.

Based on demonstration of high permeability for esmethadone HCl in this study and the demonstrated high solubility, esmethadone HCl can be classified as a BCS Class 1 drug substance.

### 3. Dissolution Study

Esmethadone Immediate Release Tablets, 25 mg were analyzed in alignment with FDA Guidance for Industry: M9 Biopharmaceutics Classification System-Based Biowaivers. Parameters for the methods used to study the dissolution are shown in Table 6, below.

TABLE 6
Summary of Dissolution Method Parameters

| | |
|---|---|
| Apparatus | USP <711> Apparatus 1 (baskets) |
| Basket speed | 100 rpm |
| Dissolution media | 0.1N HCl, Acetate buffer pH 4.5, Phosphate |
| | buffer pH 6.8 |
| Volume | 500 mL |
| Temperature | 37 ± 0.5° C. |
| Sampling time | 3, 5, 15, and 30 min. |
| Number of dosage units | 12 |
| Filter | All samples were filtered in accordance with the |
| | analytical method |
| Method | MET-000073535 |

### Summary of Dissolution Results

Results for esmethadone Immediate Release Tablets, 25 mg in dissolution media at pH 1.2, pH 4.5, and pH 6.8 demonstrated very rapid dissolution (≥85% for the mean percent dissolved in ≤15 minutes) with mean results ≥85% dissolved at all timepoints. Based on these results, calculation of the similarity factor (f2) was considered unnecessary (both test and reference products demonstrate ≥85% of the labelled amount of the drug dissolved in 15 minutes). Data shown below in Tables 7 and 8.

TABLE 7
Dissolution of Reference Drug Product: esmethadone Immediate
Release Tablet, 25 mg

| pH | % Dissolved | | | |
|---|---|---|---|---|
| (Dissolution Media) | 3 min. | 5 min. | 15 min. | 30 min. |

pH 1.2 (0.1N HCl)

| | | | | |
|---|---|---|---|---|
| Range | 89.4-106.4 | 89.8-106.9 | 89.6-108.5 | 90.1-107.3 |
| Mean (n = 12) | 100.0 | 100.0 | 100.8 | 100.7 |
| % RSD | 5.3 | 5.0 | 5.5 | 5.4 |

pH 4.5 (Acetate)

| | | | | |
|---|---|---|---|---|
| Range | 93.3-106.2 | 93.2-106.5 | 94.4-107.3 | 93.9-110.2 |
| Mean (n = 12) | 100.4 | 100.1 | 100.4 | 100.9 |
| % RSD | 4.0 | 4.3 | 4.0 | 4.9 |

pH 6.8 (Phosphate)

| | | | | |
|---|---|---|---|---|
| Range | 89.9-105.5 | 88.9-106.0 | 89.2-107.0 | 89.4-106.9 |
| Mean (n = 12) | 99.4 | 99.5 | 99.8 | 100.2 |
| % RSD | 5.1 | 5.4 | 5.9 | 5.5 |

TABLE 8
Dissolution of Test Drug Product: esmethadone Immediate Release
Tablet, 25 mg

| pH | % Dissolved | | | |
|---|---|---|---|---|
| (Dissolution Media) | 3 min. | 5 min. | 15 min. | 30 min. |

pH 1.2 (0.1N HCl)

| | | | | |
|---|---|---|---|---|
| Range | 95.5-105.0 | 97.1-106.6 | 96.0-104.7 | 95.6-106.6 |
| Mean (n = 12) | 100.7 | 101.6 | 100.9 | 101.0 |
| % RSD | 3.1 | 3.0 | 3.0 | 3.0 |

pH 4.5 (Acetate)

| | | | | |
|---|---|---|---|---|
| Range | 95.6-110.8 | 94.4-110.7 | 95.0-109.8 | 94.4-110.7 |
| Mean (n = 12) | 103.3 | 102.9 | 103.7 | 103.2 |
| % RSD | 3.8 | 4.1 | 3.7 | 3.9 |

pH 6.8 (Phosphate)

| | | | | |
|---|---|---|---|---|
| Range | 92.5-108.1 | 92.8-107.1 | 93.3-109.4 | 92.5-107.0 |
| Mean (n = 12) | 101.0 | 100.9 | 101.2 | 100.9 |
| % RSD | 4.2 | 3.5 | 4.0 | 3.5 |

### Conclusion

Both the test product formulation and the reference product formulation for esmethadone 25 mg immediate release tablets demonstrated very rapid (≥85% for the mean percent dissolved in ≤15 minutes) dissolution over the pH range of 1.2 to 6.8 with dissolution >85% released at the 3-minute timepoint.

### Example 2—Effects of Methadone and its Enantiomers on Cardiac Ion Channels

### 1. Non GLP Study of Levomethadone, Racemic Methadone and EDDP

The objective of this study was to examine the in vitro effects of three test articles on the hERG (human ether-a-go-go-related gene) channel current (a surrogate for the rapidly activating delayed rectifier cardiac potassium current, IKr). The concentration-response relationship was evaluated at near-physiological temperature (35-37° C.

Levomethadone formulation concentrations were measured on each day of testing (18-Mar-2022, 22-Mar-2022, and 24-Mar-2022). The pooled measured concentrations for the 0.3. 1, 3, and 10 μM samples were 0.366, 1.12. 3.32, and 11.33 μM, respectively. The nominal 0.3 μM results did not meet acceptance criteria (RE±15%). Measured values are therefore reported.

Racemic methadone concentrations were measured on each day of testing (30-Mar-2022 and 31-Mar-2022). The pooled measured concentrations for the 0.3. 1, 3 and 10 μM samples were 0.271, 1.00, 3.10 and 9.67 μM, respectively. These results were within the limits of the acceptance criteria demonstrating that the formulations were accurately prepared.

EDDP concentrations were measured on the day of testing (29-Mar-2022). The mean (n=2) measured for the 10 μM sample was 10.7 μM. This result was within the limits of the acceptance criteria demonstrating that the formulations were accurately prepared.

Levomethadone inhibited hERG current (Mean±SEM) by 36.0±4.9% at 0.366 μM (n=5), 54.3±6.7% at 1.13 μM (n=4), 73.6±1.5% at 3.32 μM (n=4) and 90.0±0.5% at 11.2 μM. The IC50 for the inhibitory effect of levomethadone on hERG potassium current was calculated to be 0.828 μM (Hill coefficient =0.764).

Racemie ethadone inhibited hERG current (Mean # SEM: n =4) by 33.4 =2.2% at 0.3 μM, 43.6 +4.3% at 1 uM, 71.1 +1.8% at 3 AM and 86.9 +1.0% at 10 μM. The IC50 for the inhibitory effect of racemic methadone on hERG potassium current was calculated to be 1.00 AM (Hill coefficient =0.743).

EDDP inhibited hERG current (Mean =SEM; n =3) by 43.7 +6.9% at 10.7 μM. The IC50 for the inhibitory effect of EDDP on hERG potassium current was not calculated but was estimated to be greater than 10 μM.

The positive control article verapamil inhibited hERG current (Mean±SD; n=2) by 17.4±1.6% at 0.1 μM and 83.0±0.3% at 1 μM. This result was consistent with CR-CLE historical data thereby validating the performance of the assay system.

2. GLP study of esmethadone

The in vitro effects of esmethadone on the hERO (human ether-à-go-go-related gene) channel current (a surrogate for I_{Kr}, the rapidly activating delayed rectifier cardiac potassium current) at near-physiological temperature were examined in a Good Laboratory Practice study (Charles River reference number 210112.XIJ) (incorporated by reference herein in its entirety). The methodology used followed the FDA CiPA hERG protocols published on 18-Sep-2019 (incorporated by reference herein in its entirety).

The stability of the test article formulations was confirmed during the method validation study. Test article concentrations were applied to the test system and dose solution analysis was conducted within the validated stability timeframe.

Samples for homogeneity determination were collected from the formulation. reservoirs. The sample analysis indicated that all formulations were homogeneous at the beginning of testing. Samples of the test article formulation solutions collected from the outflow of the perfusion apparatus were analyzed for concentration verification. The results from the sample analysis indicated that the measured concentrations of esmethadone at all test concentrations were within nominal concentrations; the maximum %RE was 3.7%, thereby meeting the acceptance criteria.

Esmethadone inhibited hERG current by (Mean±SEM: n=4) 27.3±1.6% at 0.3 μM, 48.8±3.4% at 1 μM. 68.6±0.3% at 3 μM, and 88.6±0.7% at 10 μM versus 9.7±4.8% (n=4) in control. hERG inhibition at 0.3, 1, 3, and 10 μM was statistically significant (P<0.05) when compared to vehicle control values. The IC50 for the inhibitory effect of esmethadone on hERG potassium current was 1.0 μM (Hill coefficient=0.8).

The positive control article verapamil inhibited hERG potassium current by (Mean±SEM; n=4) 7.5±4.2% at 0.03 μM, 23.6±3.4% at 0.1 μM, 50.0±2.6% at 0.3 μM and 83.4±0.8% at 1 μM. The IC50 for the inhibitory effect of verapamil on hERG potassium current was 0.3 μM (Hill coefficient=1.2).

### Example 3—Esmethadone Exposure and QTcF Correlation: Sub-Analysis of Data From a Phase 2 Study of Esmethadone in Patients with Major Depressive Disorder

This report details the results of the cardiodynamic evaluation of a Phase 2a, multicenter, randomized, double-blind, placebo-controlled, 3-arm study to assess the safety, tolerability, pharmacokinetic profile, and symptom response of esmethadone 25 mg QD, 50 mg QD, or placebo after a Day 1 loading dose of 75 mg, 100 mg, or placebo, respectively for 7 days, as adjunctive therapy for patients with major depressive disorder.

The primary ECG objective was to evaluate the effects of esmethadone on the QTcF interval by assessing the concentration-QT relationship using exposure-response modelling. ECG and PK time points were concentrated on Day 1. correlating with the highest exposures achieved after the initial loading dose.

The concentration-QTc and the by-time point analyses demonstrated a small effect of esmethadone on cardiac repolarization (QTcF).

The linear concentration-QTc analysis demonstrated a shallow, statistically significant, positive slope for the concentration-QTc relationship (slope 0.014 ms per ng/ml [90% CI: 0.0072 to 0.0199]) with a large and statistically significant treatment effect-specific intercept of 3.11 ms. An Eₘₐₓ model was also explored, which had overall similar results, with a slightly higher AIC and a non-significant treatment effect-specific intercept observed. A sensitivity analysis using an Eₘₐₓ model was also performed with the 5 outlier PK values in a single subject excluded, with improved performance and the smallest AIC. While the prediction of a 10 ms ΔΔQTcF threshold was similar between the models, the Eₘₐₓ models are consistent with a ΔΔQTcF plateau. The linear concentration-QTc model predicted QTcF increases of 6.7 ms (90% CI: 3.9 to 9.5) and 7.7 ms (90% CI: 4.8 to 10.6) at the geometric mean Cₘₐₓ of esmethadone after the 75 mg loading dose, 265.2 ng/ml) and after the 100 mg loading dose, 337.8 ng/ml), respectively.

The by-time point analysis also demonstrated a small, dose-dependent effect of esmethadone on QTcF, with a maximum placebo-corrected change-from-baseline QTcF of 7.7 ms (90% UCI: 3.7 to 11.7) and 10.9 ms (90% UCI: 6.2 to 15.6) in the 25 and 50 mg dose groups, respectively.

In summary, esmethadone at the studied doses produced a modest, dose-dependent increase in QTc and no clinically significant effect on heart rate, PR, or QRS.
1. Objectives and Endpoints for the ECG Analysis

### Primary Objective

The primary ECG objective was to evaluate the effects of esmethadone on the QTcF interval by assessing the concentration-QT relationship using exposure-response modelling on Day 1 for the first 23 hours after dosing.

### Secondary Objectives

The secondary ECG objectives were to assess the effect of esmethadone on other ECG parameters (HR, PR, and QRS interval), T wave morphology, the presence of pathologic U waves, and other ECG parameters (e.g., bundle branch block).

### Primary Endpoint

The primary ECG endpoint was placebo-corrected change-from-baseline QTcF (ΔΔQTcF) using the concentration-QTc analysis (primary analysis).

### Secondary Endpoints

The secondary ECG endpoints were: (1) ΔΔQTcF using the by-time point analysis; (2) Change-from-baseline HR and PR and QRS intervals (ΔHR, ΔPR, and ΔQRS) using the by-time point analysis; (3) Placebo-corrected ΔHR, ΔPR, and ΔQRS (ΔΔHR, ΔΔPR, and ΔΔQRS) using the by-time point analysis; (4) Categorical outliers for QTcF, HR, PR, and QRS; and (5) Frequency of treatment-emergent changes for T-wave morphology and U-wave presence.
2. Study Design

This was a Phase 2 study in 62 patients with a MDD randomized in a 1:1:1 ratio to receive esmethadone 25 mg QD, 50 mg QD, or placebo after a Day I loading dose of 75 mg, 100 mg, or placebo, respectively for 7 days. On Days 1 and 2, intensive ECGs were performed and on the subsequent days at 2 hours post-dose, the approximate Tₘₐₓ. Timed PK assessments were performed on Day 1 and after approximately 23 hours post Day 1 dosing, and thus for the exposure-response analysis only the Day 1 ECG data is utilized. The highest PK values were achieved on Day 1 of dosing due to the loading dose administered: 25 mg (75 mg Day 1)—254.5 ng/ml, 50 mg (100 mg Day 1)—343.9 ng/mL.

A total of 62 patients were randomized in the study, 19 patients to the esmethadone 25 mg dose group, 21 patients to the esmethadone 50 mg dose group, and 22 patients to the placebo group. Fifty-seven patients (91.9%) completed the study. Among the 5 patients who discontinued from the study early, loss to follow-up was the most commonly reported reason for early discontinuation and was reported for 3 patients (4.8%).

Safety and tolerability was assessed through end of treatment or early termination by monitoring adverse events, performing physical examinations and clinical laboratory tests, measuring vital signs, and recording ECGs.

The ECG data has previously been measured by the Core ECG Laboratory (ERT, Philadelphia, PA), but no statistical analyses, beyond descriptive statistics, including exposure-response modeling has been performed.
3. Study Population

This study enrolled patients with MDD who were between 18 and 65 years of age, inclusive. Inclusion and exclusion criteria are described by Fava and colleagues (Fava M, Stahl S. Pani L, et al. REL-1017 (Esmethadone) as Adjunctive Treatment in Patients With Major Depressive Disorder: A Phase 2a Randomized Double-Blind Trial. Am J Psychiatry. 2022;179(2): 122-131. doi:10.1176/appi.ajp.2021.21020197, which is incorporated by reference herein in its entirety).
4. ECG and Pharmacokinetic Sample Collection

The 12-lead digital ECG equipment was supplied and supported by the central ECG core lab vendor, eResearch Technology, Inc., ERT (Philadelphia, PA). After a supine rest period, ECGs were collected in triplicate at the pre-specific time points indicated within the study protocol and described below.

Twelve-lead ECGs were to be performed in triplicate approximately 1 minute apart after lying quietly for at least 3 min at:
Screening
Check-in (Day—1)
Day 1: 1 hour pre-dose and 2 hours, 4 hours, 6 hours, and 8 hours post-dose on Day 1
Day 2: 1 hour pre-dose (corresponding to 23 hours post-dose on Day 1) and 2 hours, 4 hours, 6 hours, and 8 hours post-dose
Days 3-7: 2 hours post-dose
Day 8
Prior to discharge on Day 9
End of the observation period (Day 14)

The nominal time of the ECG recordings were used for the cardiodynamic analyses.

PK sampling was done on Day 1 at 1 hour pre-dose and 0.5, 1, 2, 4, 6, 8, and 12 hours post-dose. On Day 2 through Day 7, sampling was done 1 hour pre-dose. On Day 8, sampling was done approximately 24 hours after the last dose of study drug (on Day 7). On Day 9, sampling was done approximately 48 hours after the last dose (on Day 7). Sampling was also done on Day 14 at 7 days (±3 days) (approximately 168 hours) after the last dose of study drug (on Day 7).

ECG intervals were measured by the core laboratory (ERT) in a blinded manner using the semi-automated technique. The ECG database was locked before any statistical analysis was undertaken.

The QT and RR value for each measured beat were used for HR correction. Three beats were measured on each of the triplicate ECGs at each time point. The mean value from each triplicate tracing was calculated, and then the mean of all available measurements from a nominal time point were used as the patient's reportable value at that time point.
5. ECG Methodology: Central Laboratory

ECGs were digitally transmitted to ERT for high-resolution measurement of cardiac intervals and morphological assessment by a central cardiologist blinded to the study dose. Where digital transmission was not possible, a process for receipt and analysis of scanned or paper ECGs was also established.

Digital ECGs were processed via ERT's validated data management system, EXPERT. Interval duration measurements were collected using computer-assisted caliper placement on a superimposed global median beat. A superimposed Global Median Beat was created by an algorithm, where I representative beat for each of the 12-leads was selected and superimposed. Annotation by the electrocardiograma automated algorithm was visible to the ERT technician on the computer screen. Annotations were manually adjusted by the technician wherever necessary. A cardiologist then verified the interval durations and performed the morphology analysis. On-screen measurements of the HR, PR, QRS, and QT interval durations was performed directly on the median beat and derived variables RR, QTc Fridericia and QTc Bazett (QTcF and QTcB) were calculated. Each fiducial point (onset of P wave, onset of Q wave, offset of S wave, and offset of T wave) was marked. The original ECG waveform and such annotations were saved separately in XML format for independent review.
6. Statistical Methods

All statistical analyses were performed using the statistical software SAS for Windows Version 9.4 or higher (SAS Institute, Inc., Cary, NC). Data collected from all randomized patients were presented in data listings. Both absolute values and change-from-baseline values for each patient were given where applicable. All continuous data were listed with the same precision as were presented in the database. Data listings were sorted by treatment, patient ID, and time point. Missing values were represented by an empty cell and no imputation was made.

For all descriptive statistics of continuous ECG parameters (i.e., HR, QTcF, PR, and QRS), data were summarized including number of patients (n), mean, median, standard deviation (SD), standard error (SE), 2-sided 90% confidence interval (CI), minimum, and maximum by treatment and time point. For all modeling results of the by-time-point analysis of change-from-baseline values of continuous ECG parameters, n, least squares (LS) mean, SE, and 90% CI were included. Modeling results of the by-time point analysis of placebo-corrected change-from-baseline also included LS mean, SE, and 90% CI. Mean and median values were rounded to the nearest tenth, or to the first non-zero decimal. SD, SE, and CI were rounded to the nearest hundredth. For the concentration-QTc analysis, 2 decimal places were shown for all effect estimates for all results that had an absolute value greater than 0.05. Each effect estimate with an absolute value ≤ 0.05 was displayed with 2 significant figures. The CI of the effect estimate displayed 1 more decimal place than the effect estimate. SE and P values were reported with 4 digits and P values less than 0.0001 were reported as <0.0001. Degrees of freedom (df) and t-values were reported to the nearest tenth and nearest hundredth, respectively. Percentages were rounded up or down to the nearest tenths decimal place.
7. Analysis Populations

Analysis populations for cardiodynamic ECG assessment were as defined as shown below in Table 9.

TABLE 9
Analysis populations for cardiodynamic ECG assessment

| Population | Definition |
|---|---|
| Safety population | All patients enrolled in the study who received at least |
| | 1 dose of study treatment (esmethadone or placebo) |
| PK population | All patients who received a dose of esmethadone and |
| | had at least 1 evaluable PK plasma concentration of |
| | esmethadone. |
| Cardiodynamic | All patients in the Safety population with |
| population | measurements at baseline as well as on-treatment with |
| | at least 1 post-dose time point with a valid ΔQTcF |
| | value. The Cardiodynamic population was used for the |
| | by-time point, categorical, and morphological analyses |
| | of the cardiodynamic ECG parameters. |
| C-QTc population | All patients who were in both the PK and |
| | Cardiodynamic populations with at least 1 pair of post- |
| | dose PK and ΔQTcF data from the same time point as |
| | well as patients in the Cardiodynamic population who |
| | received placebo. The C-QT population was used |
| | for the concentration-QTcF analysis. This population |
| | was defined for esmethadone. |

8. Baseline

For all continuous ECG parameters, baseline was defined as the average of the measured ECG intervals from the 1 hour pre-dose Day 1. For T-wave morphology and U-wave presence, baseline included findings observed on any of the replicates collected prior to the first dose of study drug, including screening, check-in (Day—1), unscheduled ECGs recorded prior to the first dose of study drug, and the 1 hour pre-dose Day 1.
9. Heart Rate Correction of the QT Interval and Reported QTc

Twelve-lead ECGs were collected as triplicate ECGs at each nominal time point pre-specified in the protocol, as shown in Section 4, “ECG and Pharmacokinetic Sample Collection” of this Example (above). For triplicate ECGs, the mean QTcF value across the replicate ECGs from a nominal time point was used as the patient's reportable value at that time point. QT corrected according to the Fridericia's correction (QTcF) is defined as:

$$QTcF ( ms ) = \frac{Q T ( ms )}{\sqrt[3]{\frac{R R ( ms )}{1000}}}$$

For evaluation of the HR-corrected QT interval, a scatter plot and decile plot of QTcF and RR intervals by treatment with a simple linear regression line and mean fitted regression line (90% CI) from a linear mixed-effects model, respectively were created.
10. Terminology and Definitions: Placebo-corrected ΔQTcF and Placebo-adjusted ΔQTCF (ΔΔQTcF)

Change-from-baseline QTcF (ΔQTcF) was used as the dependent variable in the by-time point analysis and in the concentration-QTc analysis.

By-time point analysis

Placebo-corrected ΔQTcF (ΔΔQTcF)

In the by-time point analysis using both descriptive analysis and statistical modeling on the QTcF interval, mean or LS mean, SE, and 2-sided 90% CI of ΔQTcF and ΔΔQTcF were calculated for each active dose group, as well as within the placebo group for ΔQTcF at each post-baseline time point.

Concentration-QTc analysis

Placebo-corrected ΔQTcF (ΔΔQTcF)

In the concentration-QTc analysis, the term placebo-corrected ΔQTcF (ΔΔQTcF) was used for the model-predicted effect across concentrations on a population level.

Definition: Model-predicted mean ΔQTcF in each active dose group minus model-predicted mean ΔQTcF in the placebo group, which equals slope estimate×concentration+treatment effect-specific intercept.

The term placebo-corrected ΔQTcF (ΔΔQTcF) was used for the model-predicted effect on the QTcF interval in the concentration-QTc prediction table and the scatter plots for concentration-QTc model(s), decile plots, and prediction plots.

### Placebo-adjusted ΔQTcF (ΔΔQTcF)

In the concentration-QTc analysis, the term placebo-adjusted ΔQTcF (ΔΔQTcF) was used to illustrate the underlying data on both patient and population levels.

Definition for the estimated placebo-adjusted ΔQTcF on a patient level: observed ΔQTcF for each patient (on active dose group or placebo group) minus the estimated time effect (i.e., the model-predicted mean ΔQTcF in the placebo group).

This term was used to illustrate the underlying data on a patient level in the scatter plot(s) for concentration-QTc model(s).

Definition for the estimated placebo-adjusted ΔQTcF term on a population level: the average of individually estimated placebo-adjusted ΔQTcF values at the associated median plasma concentration within each concentration decile.

This term was used to illustrate the underlying data on a population level in the decile plot(s).
11. Analysis

Concentration-QTc Analysis (Primary Analysis)

This analysis was performed using the C-QTc population. Unscheduled ECGs were not included in this analysis.

The relationship between plasma concentrations of esmethadone and ΔQTcF was quantified using a linear mixed-effects modeling approach using the available ECG and PK data on Day 1 through approximately 23 hours. The model had ΔQTcF as the dependent variable, plasma concentrations of esmethadone as the exploratory variate (0 for placebo), centered baseline QTcF (i.e., baseline QTcF for individual patient minus the population mean baseline QTcF for all patients) as an additional covariate, treatment (active=1 or placebo=0) and time (i.e., post-baseline time point) as fixed effects, and random effects on both intercept and slope per patient (see Garnett C, Bonate P L, Dang Q, Ferber G, Huang D, Liu J, et al. Scientific white paper on concentration-QTc modeling. [published correction appears in J Pharmacokinet Pharmacodyn. 2018;45(3):399]. J Pharmacokinet Pharmacodyn. 2018;45(3):383-397 (incorporated by reference herein in its entirety)). In all calculations, concentrations in patients who received placebo were set to zero. Plasma concentrations of esmethadone below the quantifiable limit at pre-dose were set to zero and after dosing were set to ½ the lower limit of quantitation in the concentration-QTc analysis.

The df of estimates was determined by the Kenward-Roger method, known to those of ordinary skill in the art. An unstructured covariance matrix was specified for the random effects. If convergence could not be achieved even after appropriate rescaling of the concentrations, the random effects on the slope and intercept would be dropped, in this order, until convergence was achieved. From the model, the slope (i.e., the regression parameter for concentrations of esmethadone) and the treatment effect-specific intercept (defined as the difference between active and placebo) were estimated together with the 2-sided 90% CI. The estimates for the time effect were reported with df and SE.

The geometric mean of the individual Cₘₐₓ values for patients in each of the active dose groups was determined. The model-predicted effect and its 2-sided 90% CI for ΔΔQTcF (i.e., slope estimate×concentration+treatment effect-specific intercept) at this geometric mean Cₘₐₓ were obtained. If the upper bound of the 2-sided 90% CI of the predicted effect of ΔΔQTcF at clinically relevant plasma levels of esmethadone is less than 10 ms, it will be concluded that esmethadone does not cause clinically relevant QTc prolongation within their observed plasma concentration ranges

To evaluate the adequacy of model fit with respect to the assumption of linearity, the observed ΔQTcF values adjusted by population time effect estimated from the model were used. These individual placebo-adjusted ΔQTcF_{i,k} (ΔΔQTcF_{i,k}) values equal the observed individual ΔQTcF_{i,k} for patient i administered with active drug or placebo at time point k minus the estimated population mean placebo effect at time point k (i.e., time effect). A decile plot of observed drug concentrations and the mean placebo-adjusted ΔQTcF (ΔΔQTcF) and 90% CI at the median concentration within each decile was given. The regression line presenting the model-predicted ΔΔQTcF, as described by Tornøe and colleagues (Tornøe C W, Garnett C E, Wang Y, Florian J, Li M, Gobburu J V. Creation of a knowledge management system for QT analyses. J Clin Pharmacol. 2011;51(7):1035-1042 (incorporated by reference herein in its entirety)) was added to evaluate the fit of a linear model and visualize the concentration-response relationship.

Additional exploratory analyses (via graphical displays and/or model fitting) included accounting for a delayed effect (hysteresis) and the justification for the choice of pharmacodynamic model (linear versus nonlinear)—both of which are discussed in the sections below.

### Investigation of Hysteresis

Hysteresis was assessed based on joint graphical displays of the LS mean ΔΔQTcF for each post-baseline time point from the by-time point analysis (using statistical modeling) and the geometric mean concentrations of esmethadone at the same time points. In addition, hysteresis plots were given for LS mean ΔΔQTcF and the geometric mean concentrations of esmethadone. Other concentration-QTc models such as a model with an effect compartment could be explored if all of the following 3 conditions were met:
(1) If a QT effect (ΔΔQTc) >10 ms could not be excluded in the by-time point analysis in the 25 and 50 mg dose groups (75 and 100 mg loading dose, respectively) of esmethadone with statistical modeling
(2) If the mean peak ΔΔQTcF effect was observed at the same time point for both the 25 and 50 mg doses of esmethadone in the by-time point analysis with statistical modeling
(3) If a delay between peak ΔΔQTcF and peak plasma concentrations in the plots (ΔΔQTcF versus esmethadone) of more than 1 hour was observed in a consistent way for the 25 and 50 mg doses of esmethadone

With the provision stated above, hysteresis was assumed if the curves of the hysteresis plots showed a counterclockwise loop. A significant treatment effect-specific intercept is not biologically plausible and therefore could be indicative of hysteresis or model misspecification, if it could not be explained by a nonlinear relationship.

### Appropriateness of a Linear Model

To assess the appropriateness of a linear model, normal quantile-quantile (Q-Q) plots for the standardized residuals and the random effects, scatter plots of standardized residuals versus concentration, model fitted values of ΔQTcF, and centered baseline QTcF, and box plots of standardized residuals versus nominal time and active treatment were produced. Among these plots, the scatter plots of standardized residuals versus concentration and versus centered baseline QTcF also included the LOESS (i.e., locally weighted scatter plot smoothing, as described by Cleveland (Cleveland W S. Robust locally weighted regression and smoothing scatterplots. J Am Stat Assoc. 1979;74(368):829-836 (incorporated by reference berein in its entirety)) lines with optimal smoothing parameters selected by the Akaike information criterion with a correction (Hurvich C M, Simonoff J S, and Tsai C L. Smoothing parameter selection in nonparametric regression using an improved Akaike Information Criterion. J R Stat Soc Series B Stat Methodol. 1998;60(2):271-293 (incorporated by reference herein in its entirety)). A scatter plot of observed concentrations and ΔQTcF with a LOESS line with 90% CI and a linear regression line was also provided to check the assumption of a linear concentration-QTc relationship. Since there was an indication that a linear model was potentially inappropriate, Eₘₐₓ models were also fitted. The concentration-QTc analysis was then repeated for the model found to best accommodate the potential nonlinearity detected.
12. Categorical Analysis

The categorical outlier analysis was performed using the corresponding cardiodynamic population. Unscheduled ECGs were included in this analysis.

Results for categorical outliers were summarized in frequency tables with counts and percentages for both number of patients and number of time points. Patient data were summarized using the count of distinct patients that fell into the category and the percentage of the total number of patients. Time point data were summarized using the count of time points at which the assessments fell into the category and the percentage of the total number of time points at which assessments were performed. Counts (either number of patients or number of time points) were used as the denominator in the calculation of percentages unless otherwise specified.

A patient or time point was determined as an outlier if the criteria (which were assessed separately) were met for the ECG intervals as described in Table 10.

TABLE 10
Criteria for determining a patient or time point outlier

| ECG | |
|---|---|
| interval | Categorical outlier criteria |
| QTcF | Treatment-emergent value of >450 and ≤480 ms when not |
| | present at baseline (new onset) |
| | Treatment-emergent value of >480 and ≤500 ms when not |
| | present at baseline (new onset) |
| | Treatment-emergent value of >500 ms when not present at |
| | baseline (new onset) |
| | Increase of QTcF from baseline of >30 and ≤60 ms |
| | Increase of QTcF from baseline >60 ms |
| PR | Increase of PR from baseline >25% resulting in PR >200 ms |
| QRS | Increase of QRS from baseline >25% resulting in QRS >120 ms |
| HR | Decrease of HR from baseline >25% resulting in HR <50 bpm |
| | Increase of HR from baseline >25% resulting in HR >100 bpm |

All outliers were summarized for each dose on the basis of incidence rates. A patient was counted only once for a particular outlier event if the patient experienced more than 1 episode of that event. The total number of time points was based on the number of observed time points across all patients within a dose group.
13. Morphological Analysis

Morphological analysis was performed with a focus on detecting changes in T-wave morphology and appearance of abnormal U waves. The analysis evaluated changes from baseline (i.e., treatment-emergent changes). This analysis was performed using the corresponding cardiodynamic population. Unscheduled ECGs were included in this analysis.

The analysis results for T-wave morphology and U-wave presence were summarized in frequency tables with counts and percentages for both number of patients and number of time points. The number and percentage of patients in each dose group having changes from baseline that represented the appearance of the morphological abnormality were summarized. The total number of time points having a particular change event was summarized in terms of number and percentage based on the number of observed time points across all patients within a dose group.
14. By-Time Point Analysis

In addition, the by-time point analysis of esmethadone effects on HR, PR, QRS, and QTcF was performed as secondary analyses based on cardiodynamic population. Unscheduled ECGs were not included in this analysis.

The ECG analysis was based on defining the central tendency of all ECG interval parameter changes (HR, QTcF, PR, and QRS) as a change from baseline. The baseline ECG data were compared to the post-dose ECGs.

To support the by-time point statistical modeling described below, descriptive statistics (e.g., frequency, percent, mean, SD, SE, median, maximum and minimum) were used to summarize the absolute values of the ECG interval parameters and the corresponding change-from-baseline values at each post-dose time point by dose group (including 25 mg dose of esmethadone, 50 mg dose of esmethadone, and placebo). Placebo-corrected change-from-baseline (ΔΔ) values for ECG interval parameters were also summarized using descriptive statistics for esmethadone doses groups at each post-dose time point. That is, for the placebo adjustment, the mean change-from-baseline ECG interval parameter (ΔHR, ΔQTcF, ΔPR, and ΔQRS) in the placebo group calculated at a specific time point was subtracted from individual change-from-baseline ECG for each patient on esmethadone at the same time point to generate individual placebo-corrected change-from-baseline ECG (ΔΔHR, ΔΔQTcF, ΔΔPR, and ΔΔQRS). Data-based (i.e., not model-based) 2-sided 90% CI of the mean was summarized for the change-from-baseline and placebo-corrected change-from-baseline data only.

The by-time point analysis using statistical modeling was performed as follows: The by-time point analysis for QTcF was based on a mixed-effect repeated measures model with ΔQTcF as the dependent variable, time (i.e., post-baseline time point: categorical), treatment (25 mg dose of esmethadone, 50 mg dose of esmethadone, and placebo), and time-by-treatment interaction as fixed effects, and baseline QTcF as a covariate. An unstructured covariance matrix was specified for the repeated measures at post-dose time points within patient. If the model with an unstructured covariance matrix failed to converge, other covariance matrices such as compound symmetry and autoregressive would be considered. From this analysis, the LS mean, SE, and 2-sided 90% CI were calculated for the contrast “esmethadone change from baseline” for each dose of esmethadone at each post-baseline time point, separately.

For HR, PR, and QRS intervals, the analysis was based on the post-dose ΔHR, ΔPR, and ΔQRS. The same model was used as described for the above QTc. The LS mean, SE, and 90% CI from the statistical modeling for both change-from-baseline and placebo-corrected change-from-baseline values were listed in the tables and graphically displayed.
15. Results

The concentration-QTc analyses were considered the primary analysis, while the central tendency (by-time point), categorical outlier, and morphology analyses were considered secondary. Tables (below), and the Figures of this application, detail selected data from the statistical results.

Note, reference is made throughout this Example, including all Tables and Figures, to the 25 and 50 mg dose groups for brevity. In these groups however, Day 1 dosing was 75 and 100 mg respectively (loading dose), and therefore highest exposures were achieved on Day 1, the same day the intensive PK and ECG collection was performed.

Sixty-two (62) patients were randomized in the study, 19 patients to the esmethadone 25 mg dose group, 21 patients to the esmethadone 50 mg dose group, and 22 patients to the placebo group. Fifty-seven patients (91.9%) completed the study. Among the 5 patients who discontinued from the study early, loss to follow-up was the most commonly reported reason for early discontinuation and was reported for 3 patients (4.8%) (refer to the Clinical Study Report).

Data from 19, 21, and 22 patients in the 25 and 50 mg QD esmethadone, and placebo groups, respectively are included in this analysis and report using the Cardiodynamic population. Baseline ECG parameters were similar across dose groups and within expectations for the patient population with mean HR across dose groups between 64 and 66 bpm, mean QTcF between 401 and 411 ms, mean PR between 161 and 162 ms, and mean QRS between 91 and 94 ms. The concentration-QTc analyses were based on the C-QTc population, which included 17 patients each in the 25 and 50 mg QD esmethadone dose groups.

### Effect on Cardiac Repolarization: the QTcF Interval

### By-Time Point (Central Tendency) Results

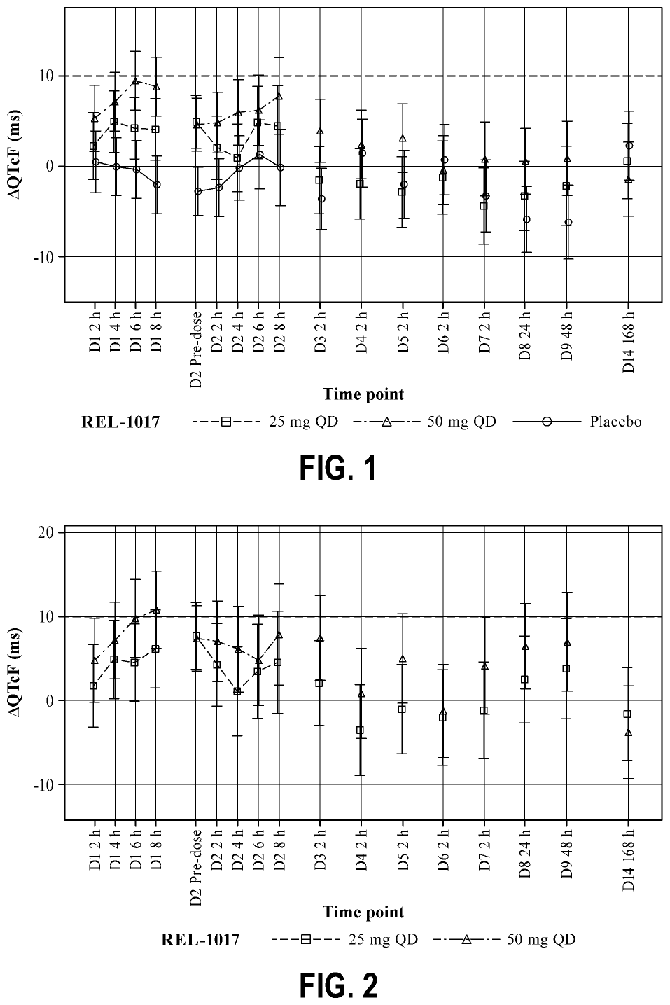

Esmethadone at the studied doses had a mild, dose-dependent QTcF prolonging effect. The LS mean change-from-baseline QTcF (ΔQTcF) values are reported below (and with reference to FIGS. 1 and 2). The LS mean ΔΔQTcF across post-dose time points on Day 1 ranged from 1.8 to 6.1 ms and from 4.9 to 10.9 ms in the 25 and 50 mg dose groups, respectively. The maximum LS mean ΔΔQTcF in the 50 mg dose group was observed at the Day 1, 8 hour time point: 10.9 ms (90% CI: 6.23 to 15.56 ms). The maximum LS mean ΔΔQTcF in the 25 mg dose group was observed at the Day 2, pre-dose time point: 7.7 ms (90% CI: 3.70 to 11.71 ms).

### Concentration-QTc Results (Linear Mixed-Effects Model)

### Pharmacokinetic Profiles

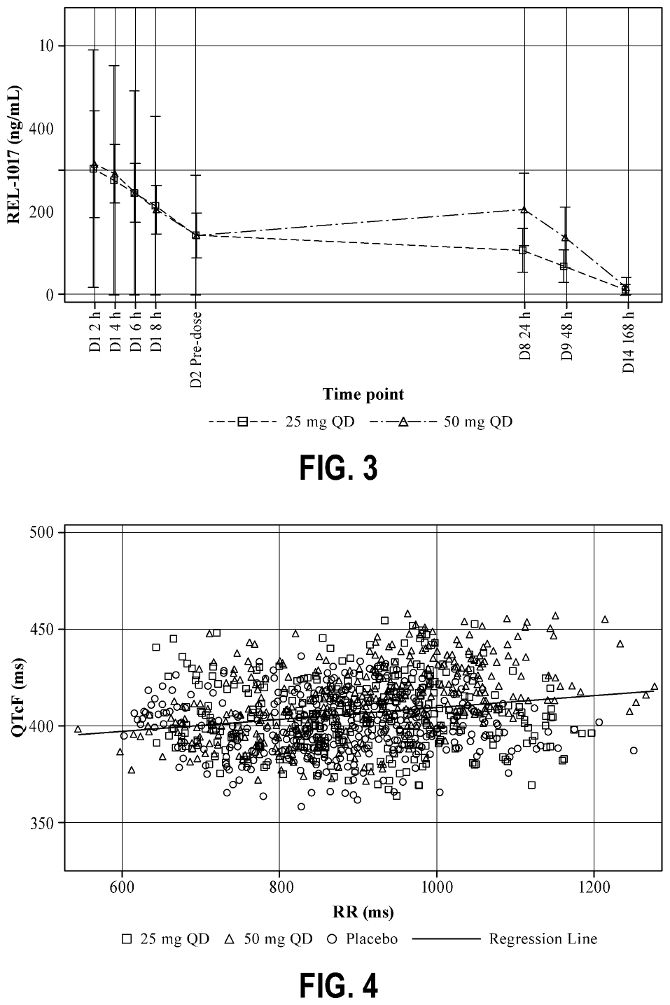

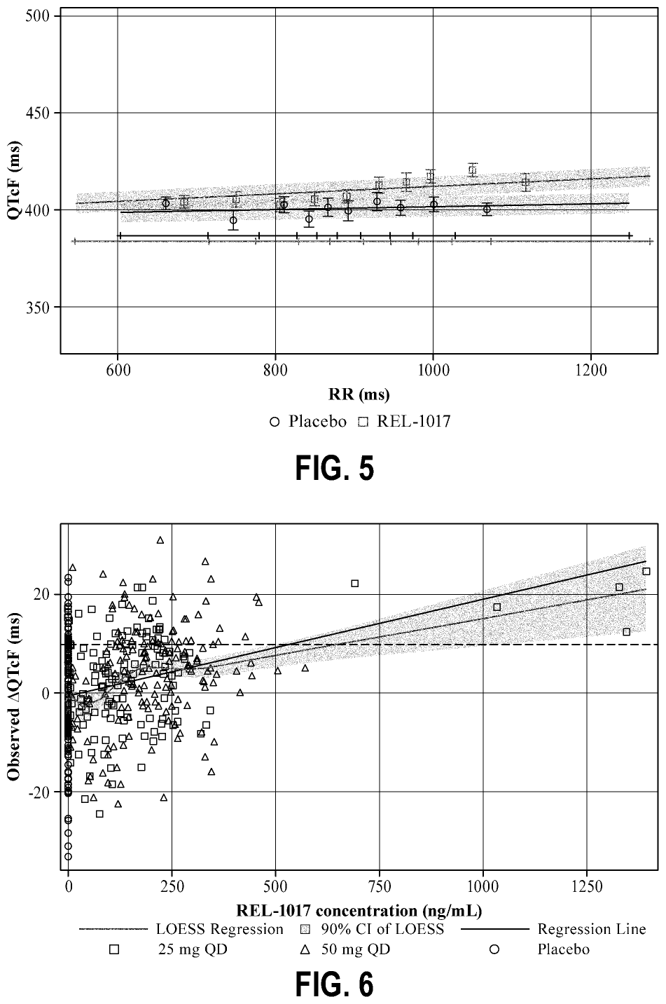

The time courses of plasma concentrations of esmethadone are shown in FIGS. 3-5.

The highest mean plasma concentrations of esmethadone were observed at 2 hour post-dose on Day 1 in both of the esmethadone 25 and 50 mg dose groups. As expected, the highest exposures followed the esmethadone loading dose on Day 1, but since only trough plasma concentrations were collected after Day 1, peak plasma concentrations following repeat dosing were not available.

### Linear Concentration-QTc Relationship

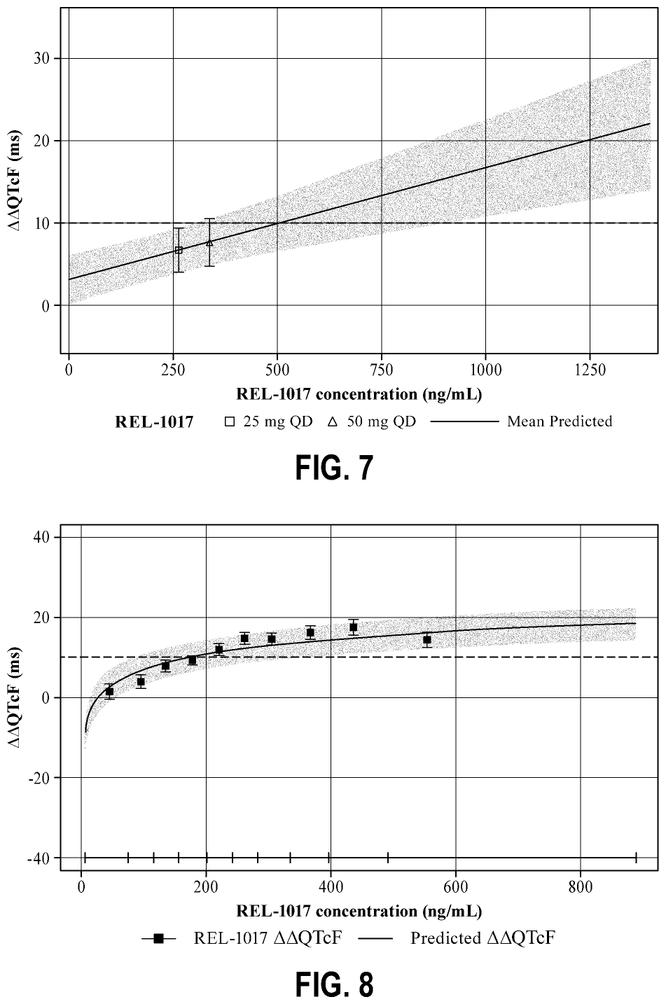

The scatter plot of concentrations of esmethadone versus ΔQTcF with a linear regression line and a LOESS regression line (90% CI) is shown in FIG. 6. The linear regression line and LOESS regression line were close to each other across the ranges of observed concentrations except at the low concentration levels. This indicates that a linear model for the concentration-QTc relationship (as shown in FIG. 7) appropriately captures the observed data.

TABLE 11
Concentration-QTc analysis of esmethadone and associated
ΔQTcF prolongation (C-QTc population)

| Parameter | Estimate | SE | df | t-Value | P Value | 90% CI |
|---|---|---|---|---|---|---|
| Treatment Effect (ms) | 3.11 | 1.7757 | 74.3 | 1.75 | 0.0835 | 0.157, 6.072 |
| esmethadone Slope (ms per ng/mL) | 0.014 | 0.0039 | 438.1 | 3.52 | 0.0005 | 0.0072, 0.0199 |
| Centered Baseline Effect (ms) | −0.17 | 0.0418 | 57.1 | −3.99 | 0.0002 | −0.236, −0.097 |
| Day 1 2 h Post-dose Effect (ms) | −1.53 | 1.6144 | 131.5 | −0.95 | 0.3458 | −4.202, 1.147 |
| Day 1 4 h Post-dose Effect (ms) | −0.21 | 1.6033 | 129.4 | −0.13 | 0.8983 | −2.861, 2.451 |
| Day 1 6 h Post-dose Effect (ms) | 0.53 | 1.5957 | 128.1 | 0.33 | 0.7401 | −2.113, 3.174 |
| Day 1 8 h Post-dose Effect (ms) | −0.20 | 1.5897 | 126.9 | −0.12 | 0.9010 | −2.832, 2.436 |
| Day 2 Pre-dose Effect (ms) | −1.38 | 1.6039 | 124.0 | −0.86 | 0.3922 | −4.035, 1.281 |
| Day 8 24 h Post-dose Effect (ms) | −6.34 | 1.6108 | 125.9 | −3.94 | 0.0001 | −9.012, −3.674 |
| Day 9 48 h Post-dose Effect (ms) | −5.44 | 1.6210 | 127.8 | −3.36 | 0.0010 | −8.128, −2.756 |
| Day 14 168 h Post-dose Effect (ms) | −1.38 | 1.6777 | 139.8 | −0.82 | 0.4127 | −4.156, 1.400 |

Based on a linear mixed-effects model with ΔQTcF as the dependent variable, time-matched esmethadone plasma concentration as an explanatory variate, centered baseline QTcF as an additional covariate, treatment (active = 1 or placebo = 0) and time as fixed effects, and a random intercept per patient.

TABLE 12
Predicted ΔΔQTcF interval at geometric mean peak esmethadone
concentrations (C-QTc population)

| Treatment | Geometric Mean (ng/ml) | ΔΔQTcF Estimate (ms) |
|---|---|---|
| Group | Cₘₐₓ of esmethadone | (90% CI) |
| 25 mg QD | 265.2 | 6.71 (3.94, 9.48) |
| 50 mg QD | 337.8 | 7.69 (4.80, 10.58) |
| 10 ms Threshold | 300 | 7.18 (4.36, 10.00) |

Based on a linear mixed-effects model with ΔQTcF as the dependent variable, time-matched esmethadone plasma concentration as an explanatory variate, centered baseline QTcF as an additional covariate, treatment (active = 1 or placebo = 0) and time as fixed effects, and a random intercept per patient.
Note:
Day 1 loading doses of 75 and 100 mg on Day 1 in the 25 and 50 mg QD cohorts, respectively, were administered.

### Summary of Concentration-QTc Results

In the concentration-QTc analysis, a linear model with a treatment effect-specific intercept was fitted for plasma concentrations; the model did not provide an optimal fit of the relationship between ΔQTcF and esmethadone plasma concentrations.

The estimated slope of the concentration-QTc relationship was positive and statistically significant (0.014 ms per ng/ml [90% CI: 0.0072 to 0.0199]) with a statistically significant intercept of 3.11 ms.

With the primary linear model, the effect on ΔQTcF is predicted to be 6.71 ms (90% CI: 3.94 to 9.48) and 7.69 ms (90% CI: 4.80 to 10.58) at the geometric mean Cₘₐₓ of esmethadone for the 25 mg cohort after a 75 mg loading dose (265.2 ng/mL) and 50 mg dose cohort after a 100 mg loading dose (337.8 ng/ml), respectively.

The goodness-of-fit plot for the linear model however suggests that the effect on ΔΔQTcF may be somewhat underestimated or overestimated at high concentrations. To explore this further, an Eₘₐₓ model was also explored. This model had an improved intercept, but slightly higher IC. A sensitivity analysis using an Eₘₐₓ model was also performed with 5 outlier PK values in a single subject excluded, with improved performance. While the prediction of a 10 ms ΔΔQTcF threshold was similar between the models, the Eₘₐₓ models are consistent with a ΔΔQTcF plateau.

Based on the linear concentration-QTc analysis, an effect on ΔQTcF with 90% UCI exceeding 10 ms is predicted to occur at an esmethadone plasma concentration of 300 ng/mL.
16. Discussion and Conclusion

esmethadone-202 was Phase 2a, multicenter, randomized, double-blind, placebo-controlled, 3-arm study to assess the safety, tolerability, PK profile, and symptom response of a 7-day dosing with esmethadone 25 mg QD and 50 mg QD as adjunctive therapy in the treatment of patients diagnosed with MDD. Patients were randomized in a 1:1:1 ratio to receive esmethadone 25 mg QD, 50 mg QD, or placebo after a Day 1 loading dose of 75 mg, 100 mg, or placebo, respectively, for 7 days. Intensive ECGs were performed on Days 1 and 2, and on the subsequent days were performed at 2 hours post-dose, which was the approximate Tₘₐₓ. Time-matched ECG-PK assessments were performed on Day 1 (2, 4, 6, 8 hours post-dose) and after approximately 23 hours post Day 1 dosing (Day 2 pre-dose time point).

The primary ECG objective was to evaluate the effects of esmethadone on the QTcF interval by assessing the concentration-QT relationship using exposure-response modelling on Day 1 for the first 23 hours after dosing.

Data were available from 19, 21, and 22 patients in the 25 and 50 mg QD

esmethadone, and placebo groups, respectively. The highest exposures were achieved on at 2 hours post-dose on Day 1 in both of the esmethadone 25 and 50 mg dose groups.

esmethadone at the studied doses had no clinically significant effect on heart rate, the PR interval, or QRS duration. There were no clinically significant ECG morphology changes.

esmethadone had a small effect on cardiac repolarization, with a modest, dose-dependent effect on QTcF. Based on the by-time point and categorical analysis, esmethadone at the studied doses had a mild, dose-dependent QT prolonging effect, with a maximum LS mean ΔΔQTcF of 7.7 ms (90% UCI: 3.7 to 11.7) and 10.9 ms (90% UCI: 6.2 to 15.6) in the 25 and 50 mg dose groups after the Day 1 loading doses, respectively (geometric mean Cₘₐₓ 265.2 ng/mL and 337.8 ng/ml, respectively).

Concentration-QTc analysis demonstrated a shallow, statistically significant, positive slope for the concentration-QTc relationship with the linear model (slope 0.014 ms per ng/ml [90% CI: 0.0072 to 0.0199]) with a large and statistically significant treatment effect-specific intercept of 3.11 ms. An Eₘₐₓ model was also explored, which had overall similar results, with a slightly higher AIC and a non-significant treatment effect-specific intercept observed. A sensitivity analysis using an Eₘₐₓ model was also performed with 5 outlier PK values in a single subject excluded, with improved performance and the smallest AIC. While the prediction of a 10 ms ΔΔQTcF threshold was similar between the models, the Eₘₐₓ models are consistent with a ΔΔQTcF plateau. The linear concentration-QTc model predicted QTcF increases of 6.7 ms (90% CI: 3.9 to 9.5) and 7.7 ms (90% CI: 4.8 to 10.6) at the geometric mean Cₘₐₓ of esmethadone for the 25 and 50 mg dose groups on Day 1, respectively.

It can be concluded that an effect on ΔQTcF with the 90% UCI exceeding 10 ms, can be excluded at the observed esmethadone plasma concentration up to 300 ng/ml. The steady state Cₘₐₓ with 25 mg oral daily dosing ×10 days is 273 ng/ml.

In summary, esmethadone at the studied doses produced a modest, dose-dependent increase in QTc and no clinically significant effect on heart rate, PR, or QRS.

Summary of sub-analysis of data from a Phase 1 study of esmethadone in healthy volunteers. (Patient population is described in Bernstein G, Davis K, Mills C, et al. Characterization of the Safety and Pharmacokinetic Profile of D-Methadone, a Novel N-Methyl-D-Aspartate Receptor Antagonist in Healthy, Opioid-Naive Subjects: Results of Two Phase 1 Studies. J Clin Psychopharmacol. 2019;39(3):226-237. doi: 10.1097/JCP.0000000000001035, incorporated by reference herein in its entirety).

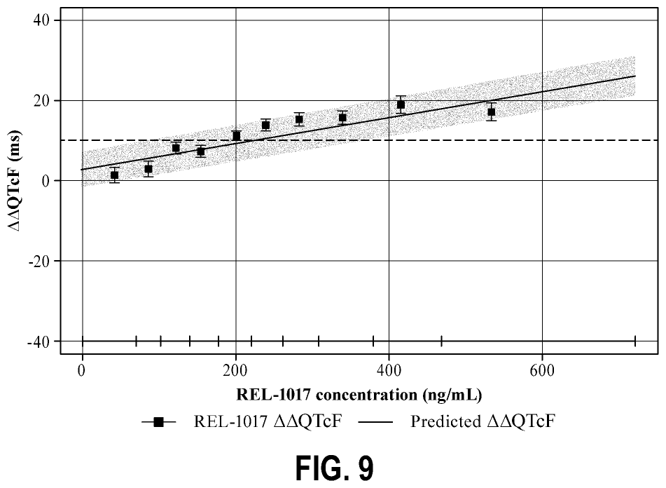

Triplicate ECGs in the esmethadone-112 study were collected time matched to PK sampling, allowing concentration-QTc modeling. Exposure-response analysis indicated that the data comparing esmethadone concentrations and the QTcF were non-linear and that log-transformation accurately modeled the data. Secondary to concentration accumulation, the QTcF effects were greater at steady state than on Day 1. The modeling confirmed a QTcF prolonging effect of esmethadone with a statistically significant slope of the relationship between plasma concentrations and QTcF. Two log-transformation models best described the data, one using all data (Model B, FIG. 8) and one excluding 3 subjects in the 50 mg group with higher PK values than in the 75 mg cohort (Model C, FIG. 9). The predicted ΔΔQTcF effect at the Day 10 steady state observed geometric mean esmethadone plasma concentration at the three doses was 11-12 ms, 16-19 ms, and 16-21 ms at the doses of 25, 50, and 75 mg QD, respectively.

The time-based central tendency analysis on Day 10, when mean esmethadone plasma concentrations were at steady state, demonstrated that the ΔΔQTcF peaked at 11.5 ms in the 25 mg cohort and at 26.8 ms and 28.8 ms in the supratherapeutic 50 mg QD and 75 mg QD dose cohorts, which are values larger than predicted with exposure-response modeling. The categorical analysis demonstrated that there were no subjects in any of the treatment groups with pronounced QTcF prolongation (i.e., QTcF>60 ms or absolute QTcF>480 ms). In addition, while a mild HR slowing was observed on Day 10 without a clear dose-dependence, there were no clinically meaningful effects on the PR or QRS intervals.

### Example 4—Methadone

This Example describes preparation and comparison of methadone tablets and modified release methadone tablets.

Preparation of tablets containing methadone 5 mg per tablet: All ingredients were first sieved on 30 mesh sieve. Lactose (17% wt/wt) and methadone (1% wt/wt) were mixed in a V-blender for about 5 minutes at 25 RPM. Microcrystalline cellulose (51% wt/wt) and pregelatizated starch (30% w/wt) were combined separately in a V-blender, blended for about 2 minutes at 25 RPM. The two mixtures were then combined, mixed in the V-Blender for minutes at 25 RPM, and then magnesium stearate (1% wt/wt) was added, and mixed for around 2 minutes at 25 RPM. This final mixture was then used to create tablets (tablet weight: 100 mg) via direct compression using a hydraulic press with 8 mm diameter die in combination with standard concave upper and lower punches.

Preparation of modified release tablets containing methadone 10 mg per tablet formulations with acrylic polymer coating: Granules of methadone were prepared by the wet granulation method after mixing the drug (2% w/wt) with the excipients: starch (62% wt/wt), pregelatinized starch (20% wt/wt), magnesium stearate (1% wt/wt), alginate (15% wt/wt). A proper amount of dry beads were compressed to obtain tablets with a specific instrument. The tablets were coated with a 5% weight gain of a retardant coating mixture including a copolymer or copolymers derived from esters of acrylic and methacrylic acids. Examples of such retardant coating mixtures include those available under the name Eudragit—and the retardant coating in this Example 4 used a mixture of Eudragit RS 30D and Eudragit RL 30D at a ratio of 90:10, RS to RL. The addition of Triethyl Citrate (a plasticizer) and Tale (anti-tacking agent) was also included in the Eudragit suspension. Once the retardant coating was complete, the tablets were given a final overcoat of a desired protective coating. The tablets were tested in a release study in vitro to demonstrate a slower drug release with respect to a fast-dissolving tablet formulation, prepared without pregelatinized starch and without coating, of the same drug (see Table 13, below).

TABLE 13
dissolution test of Example 4

| Testing sample | Time (h) | | | | | |
|---|---|---|---|---|---|---|
| Methadone 10 mg | 1 | 2 | 4 | 6 | 12 | 24 |
| Fast dissolution tablet | 70.6 | 82.4 | 97.8 | 99.8 | 100.2 | 100.1 |
| Modified release tablet | 20.5 | 36.7 | 59.8 | 72.4 | 91.2 | 96.7 |

Preparation of a modified release fornndation containing methadone 25 mg per tabler: The following materials were used in the granulation of the methadone tablets: methadone HCl, polyvinyl alcohol (PVA) and purified water. Lubricant (Compritol 888) was added to the granulation process to complete the formulation.

Methadone was loaded into the fluid bed and granulation was initiated and then then the granules were dried. Then, the granules were compressed to obtain the tablets.

The tablets were coated with a 5% weight gain of a retardant coating mixture of Eudragit RS 30D and Eudragit RL 30D at a ratio of 90:10, RS to RL. The addition of Triethyl Citrate (a plasticizer) and Talc (anti-tacking agent) was also included in the Eudragit suspension

### Example 5—Levomethadone

This Example describes preparation and comparison of levomethadone tablets and modified release levomethadone tablets.

Preparation of tablets containing levomethadone 5 mg per tablet: All ingredients were first sieved on 30 mesh sieve. Lactose (17% wt/wt) and levomethadone HCl (1% wt/wt) were mixed in a V-blender for about 5 minutes at 25 RPM. Microcrystalline cellulose (51% wt/wt) and pregelatizated starch (30% wt/wt) were combined separately in a V-blender, blended for about 2 minutes at 25 RPM. The two mixtures were then combined, mixed in the V-Blender for minutes at 25 RPM, and then magnesium stearate (1% wt/wt) was added, and mixed for around 2 minutes at 25 RPM. This final mixture was then used to create tablets (tablet weight: 100 mg) via direct compression using a hydraulic press with 8 mm diameter die in combination with standard concave upper and lower punches.

Preparation of modified release tablets containing levomethadone 10 mg per tablet formulations with acrylic polymer coating: Granules of Irvomethadone were prepared by the wet granulation method after mixing the drug (2% wt/wt) with the excipients: starch (62% wt/wt), pregelatinized starch (20% wt/wt), magnesium stearate (1% wt/wt), alginate (15% wt/wt). A proper amount of dry beads were compressed to obtain tablets with a specific instrument. The tablets were coated with a 5% weight gain of a retardant coating mixture including a copolymer or copolymers derived from esters of acrylic and methacrylic acids. Examples of such retardant coating mixtures include those available under the name Eudragit—and the retardant coating in this Example 4 used a mixture of Eudragit RS 30D and Eudragit RL 30D at a ratio of 90:10, RS to RL. The addition of Triethyl Citrate (a plasticizer) and Tale (anti-tacking agent) was also included in the Eudragit suspension. Once the retardant coating was complete, the tablets were given a final overcoat of a desired protective coating. The tablets were tested in a release study in vitro to demonstrate a slower drug release with respect to a fast-dissolving tablet formulation, prepared without pregelatinized starch and without coating, of the same drug (see Table 14, below).

TABLE 14
Dissolution test for Example 5

| Testing sample | Time (h) | | | | | |
|---|---|---|---|---|---|---|
| Levomethadone 10 mg | 1 | 2 | 4 | 6 | 12 | 24 |
| Fast dissolution tablet | 72.6 | 73.4 | 88.8 | 99.8 | 100.2 | 100.1 |
| Modified release tablet | 22.5 | 39.7 | 55.8 | 71.4 | 94.2 | 97.7 |

Preparation of modified release formulation of levomethadone at 25 mg per tablet: The following materials were used in the granulation of the levomethadone tablets: levomethadone HCl, polyvinyl alcohol (PVA) and purified water. Lubricant (Compritol 888) was added to the granulation process to complete the formulation.

Methadone was loaded into the fluid bed and granulation was initiated and then then the granules were dried. Then, the granules were compressed to obtain the tablets.

The tablets were coated with a 5% weight gain of a retardant coating mixture of Eudragit RS 30D and Eudragit RL 30D at a ratio of 90:10, RS to RL. The addition of Triethyl Citrate (a plasticizer) and Talc (anti-tacking agent) was also included in the Eudragit suspension

### Example 6—Esmethadone

This Example describes preparation and comparison of esmethadone tablets and modified release esmethadone tablets.

Preparation of tablets containing esmethadone 5 mg per tablet: All ingredients were first sieved on 30 mesh sieve. Lactose (17% wt/wt) and esmethadone HCl (1% wt/wt) were mixed in a V-blender for about 5 minutes at 25 RPM. Microcrystalline cellulose (51% wt/wt) and pregelatizated starch (30% wt/wt) were combined separately in a V-blender, blended for about 2 minutes at 25 RPM. The two mixtures were then combined, mixed in the V-Blender for minutes at 25 RPM. and then magnesium stearate (1% wt/wt) was added, and mixed for around 2 minutes at 25 RPM. This final mixture was then used to create tablets (tablet weight: 100 mg) via direct compression using a hydraulic press with 8 mm diameter die in combination with standard concave upper and lower punches.

Preparation of modified release tablets containing esmethadone 10 mg per tablet formulations with acrylic polymer coating: Granules of esmethadone were prepared by the wet granulation method after mixing the drug (2% wt/wt) with the excipients: starch (62% wt/wt), pregelatinized starch (20% wt/wt), magnesium stearate (1% wt/wt), alginate (15% wt/wt). A proper amount of dry beads were compressed to obtain tablets with a specific instrument. The tablets were coated with a 5% weight gain of a retardant coating mixture including a copolymer or copolymers derived from esters of acrylic and methacrylic acids. Examples of such retardant coating mixtures include those available under the name Eudragit—and the retardant coating in this Example 4 used a mixture of Eudragit RS 30D and Eudragit RL 30D at a ratio of 90:10, RS to RL. The addition of Triethyl Citrate (a plasticizer) and Talc (anti-tacking agent) was also included in the Eudragit suspension. Once the retardant coating was complete, the tablets were given a final overcoat of a desired protective coating. The tablets were tested in a release study in vitro to demonstrate a slower drug release with respect to a fast-dissolving tablet formulation, prepared without pregelatinized starch and without coating, of the same drug (see Table 15, below).

TABLE 15
Dissolution test of Example 6

| Testing sample | Time (h) | | | | | |
|---|---|---|---|---|---|---|
| Esmethadone 10 mg | 1 | 2 | 4 | 6 | 12 | 24 |
| Fast dissolution tablet | 71.6 | 81.4 | 97.8 | 99.8 | 100.2 | 100.1 |
| Modified release tablet | 22.5 | 38.7 | 58.8 | 70.4 | 90.2 | 98.7 |

Preparation of modified release tablets containing esmethadone at 25 mg per tablet: The following materials were used in the granulation of the esmethadone tablets: esmethadone HCl, polyvinyl alcohol (PVA) and purified water. Lubricant (Compritol 888) was added to the granulation process to complete the formulation.

Esmethadone was loaded into the fluid bed and granulation was initiated and then then the granules were dried. Then, the granules were compressed to obtain the tablets.

The tablets were coated with a 5% weight gain of a retardant coating mixture of Eudragit RS 30D and Eudragit RL 30D at a ratio of 90:10, RS to RL. The addition of Triethyl Citrate (a plasticizer) and Talc (anti-tacking agent) was also included in the Eudragit suspension

Additional examples of modified release formulations for each of esmethadone, racemic methadone, and levomethadone are reported below in Tables 16, 17, and 18:

TABLE 16
Modified release of esmethadone (25 mg per tablet)

| % w/w | mg/tablet | Ingredient name |
|---|---|---|
| 5.88% | 25.00 | Esmethadone hydrochloride |
| 23.53% | 100.00 | Methocel K4M |
| 24.71% | 105.00 | Lactose monohydrate FAST-FLO |
| 34.82% | 148.00 | Cellulose microcrystalline 102 NF/EP/JP |
| 10.00% | 42.50 | Starch 1500 |
| 0.50% | 2.13 | Colloidal silicon dioxide |
| 0.56% | 2.37 | Magnesium stearate 5712 |
| 425 mg-Theoretical core tablet weight | | |
| Coating | | |
| | 12.75 | Opadry Orange 85F13005 |
| | 72.25 mg ª | Water purified |

437.75 mg-Theoretical coated tablet weight
^{a} Removed by evaporation during the drying process.

TABLE 17
Modified release of racemic methadone (10 mg per tablet)

| % w/w | mg/tablet | Ingredient name |
|---|---|---|
| 2.35% | 10.00 | Methadone hydrochloride |
| 25.88% | 110.00 | Methocel K4M |
| 25.88% | 110.00 | Lactose monohydrate FAST-FLO |
| 34.82% | 148.00 | Cellulose microcrystalline 102 NF/EP/JP |
| 10.00% | 42.50 | Starch 1500 |
| 0.50% | 2.13 | Colloidal silicon dioxide |
| 0.56% | 2.37 | Magnesium stearate 5712 |
| 425 mg-Theoretical core tablet weight | | |
| Coating | | |
| | 12.75 | Opadry Orange 85F13005 |
| | 72.25mg ^{a} | Water purified |

437.75 mg-Theoretical coated tablet weight

TABLE 18
Modified release of levomethadone (5 mg per tablet)

| % w/w | mg/tablet | Ingredient name |
|---|---|---|
| 1.17% | 5.00 | Methadone hydrochloride |
| 25.88% | 110.00 | Methocel K4M |
| 25.88% | 110.00 | Lactose monohydrate FAST-FLO |
| 36.00% | 153.00 | Cellulose microcrystalline 102 NF/EP/JP |
| 10.00% | 42.50 | Starch 1500 |
| 0.50% | 2.13 | Colloidal silicon dioxide |
| 0.56% | 2.37 | Magnesium stearate 5712 |
| 425 mg-Theoretical core tablet weight | | |
| Coating | | |
| | 12.75 | Opadry Orange 85F13005 |
| | 72.25mg | Water purified |

437.75 mg-Theoretical coated tablet weight

While these formulation examples have been made with specific ingredients, many other ingredients may be used to achieve modified release formulations of the 3 molecules. Additional ingredients are described in the detailed description of this patent and include acrylic acid and methacrylic acid copolymers, methyl methacrylate copolymers, ethoxyethyl methacrylates. cynacethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer and glycidyl methacrylate copolymers. In certain embodiments. the acrylic polymer comprises one or more ammonio methacrylate copolymers. Ammonio methacrylate copolymers are well-known in the art, and are described in National Formulary XXI (“NF XXI”) as fully polymerized copolymers of acrylic and methacrylic acid esters with a low content of quaternary ammonium groups.

While the present invention has been disclosed by reference to the details of various embodiments of the invention, it is to be understood that the disclosure is intended as an illustrative rather than in a limiting sense, as it is contemplated that modifications will readily occur to those skilled in the art, within the spirit of the invention and the scope of the amended claims.

1. A modified release formulation of esmethadone, wherein said formulation has one or more of:
(a) a Cₘₐₓ at least 10% lower than the Cₘₐₓ of an immediate release formulation including the same dose of esmethadone,
(b) a Tₘₐₓ at least 10% longer than the Tₘₐₓ of an immediate release formulation including the same dose of esmethadone, and
(c) a Cₘᵢₙ (C_{trough}) at least 10% higher than the Cₘᵢₙ of an immediate release formulation including the same dose of esmethadone.

2. (canceled)

3. (canceled)

4. (canceled)

5. The formulation of esmethadone of claim 1 for use in patients at risk for Cₘₐₓ related side effects, Cₘₐₓ related loss of efficacy, or Cₘᵢₙ (C_{trough}) end of dose therapeutic failure.

6. The formulation of esmethadone in claim 1 for use in patients at risk for developing Cₘₐₓ related side effects including cardiac arrhythmias related to QTc prolongation.

7. The formulation of esmethadone in claim 1 for use in patients at risk for developing end of dose therapeutic failure.

8. The formulation of esmethadone in claim 1 as modified release formulations, including a hydrogel formulation.

9. The formulation of claim 1, including a 5 mg dose of esmethadone, and a Cₘₐₓ of 48 ng/ml or less.

10. The formulation of claim 1, including a 20 mg dose of esmethadone, and a Cₘₐₓ of 147 ng/ml or less.

11. The formulation of claim 2, including a 60 mg dose of esmethadone, and a Cₘₐₓ of 363 ng/ml or less.

12. The formulation of claim 2, including a 100 mg dose of esmethadone, and a Cₘₐₓ of 664 ng/ml or less.

13. The formulation of claim 2, including a 150 mg dose of esmethadone, and a Cₘₐₓ of 952 ng/ml or less.

14. The formulation of claim 2, including a 200 mg dose of esmethadone, and a Cₘₐₓ of 1377 ng/ml or less.

15. The formulation of claim 1, including a Cₘₐₓ at least 20% lower compared to an immediate release formulation.

16. The formulation of claim 15, including a 20 mg dose of esmethadone, and a Cₘₐₓ of 130.6 ng/ml or less.

17. The formulation of claim 15, including a 60 mg dose of esmethadone, and a Cₘₐₓ of 323 ng/ml or less.

18. The formulation of claim 15, including a 100 mg dose of esmethadone, and a Cₘₐₓ of 591 ng/ml or less.

19. The formulation of claim 15, including a 150 mg dose of esmethadone, and a Cₘₐₓ of 845.6 ng/ml or less.

20. The formulation of claim 15, including a 200 mg dose of esmethadone, and a Cₘₐₓ of 1224 ng/ml or less.

21. The formulation of claim 1, including a 5 mg dose of esmethadone, and a Tₘₐₓ of 165 minutes or longer.

22. The formulation of claim 1, including a 5 mg dose of esmethadone, and a Tₘₐₓ of 180 minutes or longer.

23. The formulation of claim 1, including a 25 mg dose of esmethadone, and a Cₘᵢₙ of 47.46 ng/ml or more.

24. The formulation of claim 4, including a 50 mg dose of esmethadone, and a Cₘᵢₙ of 96.36 ng/ml or more.

25. The formulation of claim 1, including a 75 mg dose of esmethadone, and a Cₘᵢₙ of 119.1 ng/ml or more.

26. The formulation of claim 1, including a 25 mg dose of esmethadone, and a Cₘᵢₙ of 51.876 ng/ml or more.

27. The formulation of claim 1, including a 50 mg dose of esmethadone, and a Cₘᵢₙ of 105.12 ng/ml or more.

28. The formulation of claim 1, including a 75 mg dose of esmethadone, and a Cₘᵢₙ of 129.96 ng/ml or more.

29. A modified release formulation of levomethadone, wherein said formulation has one or more of:
(a) a Cₘₐₓ at least 10% lower than the Cₘₐₓ of an immediate release formulation including the same dose of levomethadone.
(b) a Tₘₐₓ at least 10% longer than the Tₘₐₓ of an immediate release formulation including the same dose of levomethadone, and
(c) a Cₘᵢₙ (C_{trough}) at least 10% higher than the Cₘᵢₙ of an immediate release formulation including the same dose of levomethadone.

30. (canceled)

31. (canceled)

32. (canceled)

33. The formulation of levomethadone in claim 29 for use in patients at risk for Cₘₐₓ related side effects of Cₘₐₓ related lack of efficacy, or Cₘᵢₙ (C_{trough}) end of dose therapeutic failure.

34. The formulation of levomethadone in claim 29 for use in patients at risk for developing cardiac arrhythmias, sedation, respiratory depression, euphoria.

35. The formulation of levomethadone in claim 29 for use in patients with opioid use disorder at risk for relapse.

36. We claim the formulation of levomethadone in claim 29 for use in patients with difficult to control pain at risk for breakthrough pain.

37. The formulation of levomethadone in claim 29 as a hydrogel modified release formulation.

38. The formulation of claim 29, including a 5 mg dose of levomethadone, and a Cₘₐₓ of 48 ng/ml or less.

39. The formulation of claim 29, including a 20 mg dose of levomethadone, and a Cₘₐₓ of 147 ng/ml or less.

40. The formulation of claim 29, including a 60 mg dose of levomethadone, and a Cₘₐₓ of 363 ng/ml or less.

41. The formulation of claim 29, including a 100 mg dose of levomethadone, and a Cₘₐₓ of 664 ng/ml or less.

42. The formulation of claim 29, including a 150 mg dose of levomethadone, and a Cₘₐₓ of 952 ng/ml or less.

43. The formulation of claim 29, including a 200 mg dose of levomethadone, and a Cₘₐₓ of 1377 ng/ml or less.

44. The formulation of claim 29, including a Cₘₐₓ at least 20% lower compared to an immediate release formulation.

45. The formulation of claim 44, including a 20 mg dose of levomethadone, and a Cₘₐₓ of 130.6 ng/ml or less.

46. The formulation of claim 44, including a 60 mg dose of levomethadone, and a Cₘₐₓ of 323 ng/ml or less.

47. The formulation of claim 44, including a 100 mg dose of levomethadone, and a Cₘₐₓ of 591 ng/ml or less.

48. The formulation of claim 44, including a 150 mg dose of levomethadone, and a Cₘₐₓ of 845.6 ng/ml or less.

49. The formulation of claim 44, including a 200 mg dose of levomethadone, and a Cₘₐₓ of 1224 ng/ml or less.

50. The formulation of claim 29, including a 5 mg dose of levomethadone, and a Tₘₐₓ of 165 minutes or longer.

51. The formulation of claim 29, including a 5 mg dose of levomethadone, and a Tₘₐₓ of 180 minutes or longer.

52. The formulation of claim 29, including a 25 mg dose of levomethadone, and a Cₘᵢₙ of 47.46 ng/ml or more.

53. The formulation of claim 29, including a 50 mg dose of levomethadone, and a Cₘᵢₙ of 96.36ng/ml or more.

54. The formulation of claim 29, including a 75 mg dose of levomethadone, and a Cₘᵢₙ of 119.1 ng/ml or more.

55. The formulation of claim 29, including a 25 mg dose of levomethadone, and a Cₘᵢₙ of 51.876 ng/ml or more.

56. The formulation of claim 29, including a 50 mg dose of levomethadone, and a Cₘᵢₙ of 105.12 ng/ml or more.

57. The formulation of claim 29, including a 75 mg dose of levomethadone, and a Cₘᵢₙ of 129.96 ng/ml or more.

58. A modified release formulation of racemic methadone, wherein the formulation has one or more of:
(a) a Cₘₐₓ at least 10% lower than the Cₘₐₓ of an immediate release formulation including the same dose of racemic methadone,
(b) a Tₘₐₓ at least 10% longer than the Tₘₐₓ of an immediate release formulation including the same dose of racemic methadone, and
(c) a Cₘᵢₙ (C_{trough}) at least 10% higher than the Cₘᵢₙ of an immediate release formulation including the same dose of racemie methadone.

59. (canceled)

60. (canceled)

61. (canceled)

62. The formulation of racemic methadone in claims 58 for use in patients at risk for Cₘₐₓ related side effects or Cₘₐₓ related lack of efficacy of Cₘᵢₙ (C_{trough}) end of dose therapeutic failure.

63. The formulation in claims 58 for use in patients at risk for developing cardiac arrhythmias, sedation, respiratory depression, euphoria.

64. The formulation of claims 58 for use in patients with opioid use disorder at risk for relapse.

65. The formulation of claims 58 for use in patients with difficult to control pain at risk for breakthrough pain.

66. The formulation of claims 58 as a hydrogel modified release formulation.

67. The formulation of claim 58, including a 5 mg dose of racemic methadone, and a Cₘₐₓ of 48 ng/ml or less.

68. The formulation of claim 58, including a 20 mg dose of racemic methadone, and a Cₘₐₓ of 147 ng/ml or less.

69. The formulation of claim 58, including a 60 mg dose of racemic methadone, and a Cₘₐₓ of 363 ng/ml or less.

70. The formulation of claim 58, including a 100 mg dose of racemic methadone, and a Cₘₐₓ of 664 ng/ml or less.

71. The formulation of claim 58, including a 150 mg dose of racemic methadone, and a Cₘₐₓ of 952 ng/ml or less.

72. The formulation of claim 58, including a 200 mg dose of racemic methadone, and a Cₘₐₓ of 1377 ng/ml or less.

73. The formulation of claim 58, including a Cₘₐₓ at least 20% lower compared to an immediate release formulation.

74. The formulation of claim 73, including a 20 mg dose of racemic methadone, and a Cₘₐₓ of 130.6 ng/ml or less.

75. The formulation of claim 73, including a 60 mg dose of racemic methadone, and a Cₘₐₓ of 323 ng/ml or less.

76. The formulation of claim 73, including a 100 mg dose of racemic methadone, and a Cₘₐₓ of 591 ng/ml or less.

77. The formulation of claim 73, including a 150 mg dose of racemic methadone, and a Cₘₐₓ of 845.6 ng/ml or less.

78. The formulation of claim 73, including a 200 mg dose of racemic methadone, and a Cₘₐₓ of 1224 ng/ml or less.

79. The formulation of claim 58, including a 5 mg dose of racemic methadone, and a Tₘₐₓ of 165 minutes or longer.

80. The formulation of claim 58, including a 5 mg dose of racemic methadone, and a Tₘₐₓ of 180 minutes or longer.

81. The formulation of claim 58, including a 25 mg dose of racemic methadone, and a Cₘᵢₙ of 47.46 ng/ml or more.

82. The formulation of claim 58, including a 50 mg dose of racemic methadone, and a Cₘᵢₙ of 96.36 ng/ml or more.

83. The formulation of claim 58, including a 75 mg dose of racemic methadone, and a Cₘᵢₙ of 119.1 ng/ml or more.

84. The formulation of claim 58, including a 25 mg dose of racemic methadone, and a Cₘᵢₙ of 51.876 ng/ml or more.

85. The formulation of claim 58, including a 50 mg dose of racemic methadone, and a Cₘᵢₙ of 105.12 ng/ml or more.

86. The formulation of claim 58, including a 75 mg dose of racemic methadone, and a Cₘᵢₙ of 129.96 ng/ml or morc.

87. A method comprising:
obtaining the Cₘₐₓ, Tₘₐₓ, and Cₘᵢₙ for an oral immediate release formulation of racemic methadone, levomethadone, or esmethadone in a patient, and
subsequently administering an oral modified release formulation of racemie methadone. levomethadone, or esmethadone that results in at least 10% lower Cₘₐₓ, at least 10% delayed Tₘₐₓ and at least 10% higher Cₘᵢₙ compared to the Cₘₐₓ, Tₘₐₓ, and Cₘᵢₙ that was obtained for the oral immediate release formulation.

88. A method comprising:
obtaining the Cₘₐₓ for an oral immediate release formulation of racemic methadone, levomethadone, or esmethadone in a patient, and
subsequently administering an oral modified release formulation of racemic methadone, levomethadone, or esmethadone that results in at least 10% lower Cₘₐₓ compared to the Cₘₐₓ that was obtained for the oral immediate release formulation.

89. A method comprising:
obtaining the Tₘₐₓ for an oral immediate release formulation of racemic methadone, levomethadone, or esmethadone in a patient, and
subsequently administering an oral modified release formulation of racemic methadone, levomethadone, or esmethadone that results in at least 10% delayed Tₘₐₓ compared to the Tₘₐₓ that was obtained for the oral immediate release formulation.

90. A method comprising:
obtaining the Cₘᵢₙ for an oral immediate release formulation of racemic methadone, levomethadone, or esmethadone in a patient, and
subsequently administering an oral modified release formulation of racemic methadone, levomethadone, or esmethadone that results in at least 10% higher Cₘᵢₙ compared to the Cₘᵢₙ that was obtained for the oral immediate release formulation.
